(19) United States
(12) Patent Application Publication
(10) Pub. No.: US 2003/0055263 A1
(43) Pub. Date: Mar. 20, 2003

(54) Carboxylic acid derivatives, medicaments comprising these compounds, their use and processes for their production

(75) Inventors: Henning Priepke, Warthausen (DE); Iris Kauffmann-Hefner, Attenweiler (DE); Norbert Hauel, Schemmerhofen (DE); Klaus Damm, Biberach (DE); Andreas Schnapp, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/192,456

(22) Filed: Jul. 10, 2002

Related U.S. Application Data
(60) Provisional application No. 60/307,449, filed on Jul. 24, 2001.

(30) Foreign Application Priority Data
Jul. 11, 2001 (DE) 101 33 665.9

(51) Int. Cl. C07D 277/62; C07D 263/60; C07D 235/26; C07C 69/76

(52) U.S. Cl. 548/170; 548/217; 548/309.4; 548/494; 560/103

(57) ABSTRACT

The present application relates to the use of the carboxylic acid derivatives of general formula

R₁—A—B—R₂ (I)

wherein

R₁, R₂, A and B are defined as in claim 1, the isomers and the salts thereof, particularly the physiologically acceptable salts thereof, which have an inhibitory effect on telomerase, processes for the preparation thereof, pharmaceutical compositions containing these compounds and the use thereof as well as the preparation thereof.

### CROSS REFERENCE TO RELATED APPLICATION

This application claims the benefit of U.S. provisional application No. 60/307,449 filed Jul. 24, 2001. The contents of which are fully incorporated by reference herein.

### FIELD OF THE INVENTION

The present invention relates to carboxylic acid derivatives and medicaments thereof their production and processes and their use in the inhibiting effect on telemerase and oncogenesis.

### BACKGROUND OF THE INVENTION

The last decade of oncological research has made it possible for the first time to achieve a molecular understanding of the regulatory mechanisms involved in the formation of tumours. These include, for example, the function of oncogenes, tumour suppressor genes, growth factors, receptors, signal transduction cascades, pro- and anti-apoptotic genes in controlling cell growth, differentiation, migration and cell death. These new findings have also shown, however, that cancer is a multifactorial disease at the molecular level, during the onset of which tissues may undergo malignant degeneration as a result of different mechanisms. This heterogeneity of the malignant cells in turn explains the clinical problems of tumour therapy.

Back in 1965 Hayflick postulated (Hayflick, Exp. Cell Res. 37, 614-636 (1965)) that the limited proliferative lifespan of normal somatic cells, replicative senescence, can act as a tumour suppressing mechanism. This hypothesis was supported by experimental work which showed that the overcoming of replicative senescence is a prerequisite for the malignant transformation of cells (Newbold et al. in Nature, 299, 633-636 (1989); Newbold and Overell in Nature, 304, 648-651 (1983)).

However, only in the last few years has there been any understanding of the molecular mechanisms by which somatic cells achieve the state of replicative senescence.

The ends of eukaryotic chromosomes, the telomers, consist of simple repetitive sequences the integrity of which is essential for the function and structure of the chromosomes. However, linear chromosomes lose a certain length of their telomers in each round of DNA replication, a phenomenon which was recognised by Watson back in 1972 (Watson in Nature New Biol. 239, 197-201 (1972)). The cumulative loss of telomeric DNA over numerous cell divisions constitutes the basis for the limited replicative potential of somatic cells, whereas more than 85% of all tumours in humans reactivate an enzyme, telomerase, in order to compensate for the loss of telomers and thus become immortal (see Shay and Bacchetti in European Journal of Cancer, 33, 787-791 (1997)).

Telomerase in humans is a ribonucleoprotein (RNP) which is made up of at least one catalytic subunit (hTERT), and one RNA (hTR). Both components have been molecularly cloned and characterised. Biochemically, telomerase is a reverse transcriptase which uses a sequence fragment in hTR as matrix in order to synthesise a strand of telomeric DNA (Morin in Cell 59, 521-529 (1989)). Methods of identifying telomerase activity and also methods of diagnosing and treating replicative senescence and immortality by modifying telomers and telomerase have already been described (Morin in Cell 59, 521-529 (1989); Kim et al. in Science 266, 2011-2014 (1994)).

Inhibitors of telomerase can be used for tumour therapy since somatic cells, unlike tumour cells, are not dependent on telomerase.

### SUMMARY OF THE INVENTION

It has now been found that the carboxylic acid derivatives of general formula

R₁—A—B—R₂ (I)

the isomers and the salts thereof, particularly the physiologically acceptable salts thereof, have an inhibiting effect on telomerase.

In the above general formula I

R₁ denotes a phenyl, phenyl-C₁₋₃-alkyl, phenyl-C₂₋₄-alkenyl or naphthyl group, wherein in each case the aromatic moieties may be mono- or disubstituted by a fluorine, chlorine, bromine or iodine atom, by a C₁₋₃-alkyl or C₁₋₃-alkoxy group, while the substituents may be identical or different,

a phenyl group, to which an n-propylene, n-butylene, methylenedioxy or ethylenedioxy bridge is fused via two adjacent carbon atoms,

a phenyl group, to which a 5-membered heteroaromatic group is fused via two adjacent carbon atoms, which contains, in the heteroaromatic moiety,

an imino group optionally substituted by a C₁₋₃-alkyl group, an oxygen or sulphur atom,

an imino group optionally substituted by a C₁₋₃-alkyl group and an oxygen, sulphur or nitrogen atom,

an imino group optionally substituted by a C₁₋₃-alkyl group and two nitrogen atoms or

an oxygen or sulphur atom and two nitrogen atoms,

a pyridinyl or pyronyl group optionally substituted by a C₁₋₃-alkyl group, to which a phenyl ring may be fused in each case via two adjacent carbon atoms, while in the abovementioned pyridine ring additionally a methine group in the 2 or 4 position may be replaced by a hydroxymethine group,

A denotes a phenylene group optionally substituted by a C₁₋₃-alkyl group, wherein in the aromatic moiety one, two or three methine groups may be replaced by nitrogen atoms, or

a 5-membered heteroarylene group optionally substituted by a C₁₋₃-alkyl group, while the heteroaromatic moiety is as hereinbefore defined,

B denotes an —HN, —NH—CO, —CO—NH, —NH—CS or —CS—NH group, wherein the —NH group may be substituted in each case by a C₁₋₃-alkyl group, and

R₂ denotes a C₃₋₇-cycloalkyl or C₄₋₇-cycloalkenyl group substituted by a carboxy group,

a phenyl or naphthyl group substituted by a carboxy group, wherein in each case the aromatic moiety may be replaced by a nitro, amino, C₁₋₃-alkylamino, di-(C₁₋₃-alkyl)-amino, C₁₋₃-alkanoylamino, N-(C₁₋₃-alkyl)-C₁₋₃-alkanoylamino or carboxy group, by an aminocarbonyl or C₁₋₃-alkylaminocarbonyl group, wherein in each case the hydrogen atom of the aminocarbonyl group is monosubstituted by a C₁₋₃-alkyl or C₃₋₇-cycloalkyleneimino group, or is mono- or disubstituted by a fluorine, chlorine, bromine or iodine atom, by a C₁₋₃-alkyl or C₁₋₃-alkoxy group, while the substituents may be identical or different,

a 5- or 6-membered heteroaryl group substituted by a carboxy group, while the 5-membered heteroaryl group is as hereinbefore defined and

the 6-membered heteroaryl group contains one or two nitrogen atoms,

or a straight-chain or branched C₁₋₆-alkyl or C₂₋₆-alkenyl group substituted by a carboxy group,

while the carboxy groups mentioned in the definition of the abovementioned groups may additionally be replaced by a group which is converted in viVo into a carboxy group.

By a group which can be converted in vivo into a carboxy group is meant, for example, a hydroxmethyl group, a carboxy group esterified with an alcohol, wherein the alcoholic moiety preferably denotes a C₁₋₆-alkanol, a phenyl-C₁₋₃-alkanol, a C₃₋₉-cycloalkanol, whilst a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol having a total of 8 to 10 carbon atoms which may additionally be substituted by one or two C₁₋₃-alkyl groups in the bicycloalkyl moiety, a 1,3-dihydro-3-oxo-1-isobenzofuranol or an alcohol of formula

Rₐ—CO—O—(R_{b}CR_{c})—OH,

wherein

Rₐ denotes a C₁₋₈-alkyl, C₅₋₇-cycloalkyl, phenyl or phenyl-C₁₋₃-alkyl group,

R_{b} denotes a hydrogen atom, a C₁₋₃-alkyl, C₅₋₇-cycloalkyl or phenyl group and

R_{c} denotes a hydrogen atom or a C₁₋₃-alkyl group.

By a group which is negatively charged under physiological conditions is meant a carboxy, hydroxysulphonyl, phosphono, tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonyl-aminocarbonyl group

and by a group which can be cleaved in vivo from an imino or amino group is meant, for example, a hydroxy group, an acyl group such as the benzoyl or pyridinoyl group or a C₁₋₁₆-alkanoyl group such as the formyl, acetyl, propionyl, butanoyl, pentanoyl or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert. butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or Rₐ—CO—O—(R_{b}CR_{c})—O—CO group wherein Rₐ to R_{c} are as hereinbefore defined.

Moreover, the saturated alkyl and alkoxy moieties containing more than 2 carbon atoms mentioned in the definitions given above also include the branched isomers thereof, such as the isopropyl, tert.butyl, isobutyl group, etc.

Preferred compounds of general formula I are those wherein

R₁ denotes a phenyl group which may be substituted by a chlorine, bromine or iodine atom, may be mono- or disubstituted by a methyl or methoxy group, while the substituents may be identical or different,

a phenylvinyl, benzothiophenyl or naphthyl group,

a phenyl group to which an n-propylene, n-butylene, methylenedioxy or ethylenedioxy bridge is fused via two adjacent carbon atoms,

an pyridinyl or pyronyl group optionally substituted by a methyl group, to which a phenyl ring is fused in each case via two adjacent carbon atoms, while in the abovementioned pyridine ring a methine group in the 2 or 4 position may additionally be replaced by a hydroxymethine group,

A denotes a phenylene, furanylene, thiophenylene, thiazolylene, imidazolylene, thiadiazolylene, pyridinylene or pyrimidylene group optionally substituted by a methyl group with the proviso that linking to the adjacent groups R₁ and B does not take place via the o position of the abovementioned aromatic groups,

B denotes an —HN, —NH—CO, —CO—NH, —NH—CS or —CS—NH group, wherein the —NH group may be substituted in each case by a methyl group, and

R₂ denotes a C₃₋₆-cycloalkyl or C₄₋₆-cycloalkenyl group substituted by a carboxy group,

a phenyl group substituted by a carboxy group which is monosubstituted in the phenyl moiety by a nitro, amino, acetylamino, carboxy, aminocarbonyl or

pyrrolidinoaminocarbonyl group or is mono- or disubstituted by a fluorine, chlorine, bromine or iodine atom or by a methyl or methoxy group, while the substituents may be identical or different,

a carboxy-substituted naphthyl, furanyl, thiophenyl, triazolyl or pyridinyl group,

an aminocarbonylmethyl group or a carboxy-substituted methyl or 1,2-dimethylvinyl group,

the isomers and the salts thereof.

Particularly preferred compounds of general formula I are those wherein in each case R₁, R₂ and A are as hereinbefore defined and

B denotes an —NH or —NH—CO group, while the —NH—CO group is linked to the group R₂ via the —CO group,

the isomers and the salts thereof.

Most particularly preferred compounds of the above general formula I are those wherein

R₁ denotes a phenyl group optionally mono- or disubstituted by a chlorine, bromine or iodine atom, while the substituents may be identical or different,

a naphthyl or (2-oxo-2H-chromen-3-yl) group,

A denotes a 1,3-phenylene, 2,5-thiazolylene, 2,4-pyridinylene, 2,6-pyridinylene or 2,4-pyrimidylene group,

B denotes an —NH or —NH—CO group, while the —NH—CO group is linked to the group R₂ via the —CO group,

R₂ denotes a 2-carboxy-cyclopent-2-enyl, 2-carboxy-cyclohex-2-enyl, 3-carboxy-thien-2-yl or 2-carboxy-1,2-dimethyl-vinyl group or

a 2-carboxy-phenyl group optionally monosubstituted by a fluorine, chlorine or bromine atom or by a methyl or nitro group,

the isomers and the salts thereof.

The following are mentioned as examples of particularly preferred compounds of the above general formula I:

(a) 2-[4-(naphthalin-2-yl)-thiazol-2-ylaminocarbonyl]-benzoic acid,

(b) 2-[4-(naphthalin-2-yl)-thiazol-2-ylaminocarbonyl]-cyclopent-1-ene-carboxylic acid and

(c) 2-[4-(naphthalin-2-yl)-pyrimidin-2-ylamino]-benzoic acid

and the salts thereof.

### DETAILED DESCRIPTION OF THE INVENTION

The carboxylic acid amide of the above general formula I is obtained for example by the following methods known per se:

a. reacting a compound of general formula

R₁—A—U (II)

with a compound of general formula

V—R₂ (III)

wherein

R₁, R₂ and A are as hereinbefore defined,

one of the groups U or V denotes an amino group optionally substituted by a C₁₋₃-alkyl group and

the other of the groups U or V represents a carboxy group, or the reactive derivatives thereof.

The reaction is conveniently carried out with a corresponding halide or anhydride in a solvent such as methylene chloride, chloroform, carbon tetrachloride, ether, tetrahydrofuran, dioxane, benzene, toluene, acetonitrile, dimethylformamide, dimethylsulphoxide or sulpholane, optionally in the presence of an inorganic or tertiary organic base such as triethylamine, N-ethyl-diisopropylamine, N-methyl-morpholine or pyridine, while the latter may also be used as the solvent at the same time, at temperatures between −20 and 200° C., but preferably at temperatures between −10 and 160° C.

However, the reaction may also be carried out with a free acid, optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

b. In order to prepare a compound of general formula I wherein R₂ is as hereinbefore defined, with the proviso that the carboxy group of the group R₂ is in the 2 position, and B denotes an —NHCO group, while the carbonyl group of the group B is linked to the group R₂:

hydrolysis of a compound of general formula

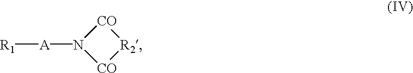

or of general formula

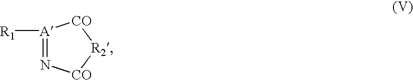

wherein

R₁ and A are as hereinbefore defined,

R₂′ has the meanings given for R₂ hereinbefore, with the proviso that the carbonyl group of the group R₂ originating from the carboxy substituent is in the 2 position, and A′ has the meanings given for A hereinbefore, with the proviso that A contains a nitrogen atom, which is linked to the carbonyl group.

The hydrolysis is conveniently carried out either in the presence of an acid such as hydrochloric acid, sulphuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxane at temperatures between −10 and 120° C., e.g. at temperatures between ambient temperature and the boiling temperature of the reaction mixture.

c. In order to prepare a compound of general formula I wherein B denotes an —NH group optionally substituted by a C₁₋₃-alkyl group:

reacting a compound of general formula

R₁—A—X (VI),

with a compound of general formula

Y—R₂ (VII),

wherein

R₁, R₂ and A are as hereinbefore defined,

one of the groups X or Y denotes an amino group optionally substituted by a C₁₃-alkyl group and

the other of the groups X or Y denotes a leaving group such as a substituted sulphonyloxy group or a halogen atom, e.g. a trifluoromethylsulphonyloxy group, a chlorine, bromine or iodine atom.

The reaction is carried out at elevated temperatures, conveniently in a solvent such as ethanol, dimethoxyethane, tetrahydrofuran, acetonitrile, toluene or xylene, e.g. at the boiling temperature of the solvent used, and preferably in the presence of a reaction accelerator such as concentrated hydrochloric acid, 2,2′-bis-(diphenylphosphino)-1,1′-binaphthyl/palladium acetate, palladium-tetrakistriphenylphosphine/2,2′-bis(diphenylphosphio)-1,1′-binaphthyl or catalysts, such as those described for example in Angew. Chemie Int. Ed. Engl. 37, 2090 (1998), in the presence of a base such as caesium carbonate, sodium or potassium tert. butoxide.

d. In order to prepare a compound of general formula I wherein B denotes an —NHCS or —CS—NH group optionally substituted at the amide nitrogen atom by a C₁₋₃-alkyl group:

reacting a compound of general formula

R₁—A—B—R₂ (VIII),

wherein

R₁, R₂ and A are as hereinbefore defined and

B′ denotes an —NH—CO or —CO—NH group optionally substituted at the amide nitrogen atom by a C₁₋₃-alkyl group, with a sulphurising agent.

The reaction is carried out in the presence of a sulphurising agent such as for example 2,4-bis(4-methoxyphenyl)-1,3-dithia-2,4-diphosphetan-2,4-disulphide (Lawesson's reagent) or phosphorus pentasulphide conveniently in a solvent such as tetrahydrofuran, dioxane, toluene, xylene, 1,2-dichlorobenzene or pyridine at temperatures up to the boiling temperature of the solvent used, e.g. at temperatures between 20 and 180° C.

e. In order to prepare a compound of general formula I wherein R₂ has the meanings given for R₂ hereinbefore with the proviso that the carboxy substituent is replaced by a group which may be converted in vivo into a carboxy group:

reacting a compound of general formula

R₁—A—B—R₂″ (IX),

wherein

R₁, A and B are as hereinbefore defined and

R₂″ has the meanings given for R₂ hereinbefore with the proviso that it is substituted by a carboxy group, or the alkali metal salt thereof, with a compound of general formula

Z—R (X),

wherein

R denotes a C₁₋₆-alkyl, phenyl-C₁₋₃-alkyl or C₃₋₉-cycloalkyl group, while the C₅₋₈-cycloalkyl moiety may additionally be substituted by one or two C₁₋₃-alkyl groups, a C₅₋₈-cycloalkyl group wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkyl moiety may additionally be substituted by one or two C₁₋₃-alkyl groups, a C₄₋₇-cycloalkenyl, C₃₋₅-alkenyl, phenyl-C₃₋₅-alkenyl, C₃₋₅-alkynyl or phenyl-C₃₋₅-alkynyl group with the proviso that no bond to the oxygen atom starts from a carbon atom that carries a double or triple bond, a C₃₋₈-cycloalkyl-C₁₋₃-alkyl group, a bicycloalkyl group with a total of 8 to 10 carbon atoms, while the bicycloalkyl moiety may additionally be substituted by one or two C₁₋₃-alkyl groups, a 1,3-dihydro-3-oxo-1-isobenzofuranyl group or a group of general formula

Rₐ—CO—O—(R_{b}CR_{c})—,

wherein

Rₐ to R_{c} are as hereinbefore defined,

and Z represents a nucleofugic leaving group such as a halogen atom, e.g. a chlorine, bromine or iodine atom, a hydroxy or p-nitrophenyloxy group.

The conversion of a carboxy group into a group which may be converted into a carboxy group in vivo is preferably carried out by esterification with a corresponding alcohol or by alkylation of the carboxy group. The esterification is conveniently carried out in a solvent or mixture of solvents such as methylene chloride, benzene, toluene, chlorobenzene, tetrahydrofuran, benzene/tetrahydrofuran or dioxane, but preferably in an excess of the alcohol used in the presence of a dehydrating agent, e.g. in the presence of hydrochloric acid, sulphuric acid, isobutyl chloroformate, thionyl chloride, trimethylchlorosilane, hydrochloric acid, sulphuric acid, methanesulphonic acid, p-toluenesulphonic acid, phosphorus trichloride, phosphorus pentoxide, 2-(1H-benzotriazol-1-yl)-1,1,3,3-tetramethyluronium-tetrafluoroborate, N,N′-dicyclohexylcarbodiimide, N,N′-dicyclohexylcarbodiimide/N-hydroxysuccinimide, N,N′-carbonyldiimidazole or N,N′-thionyldiimidazole, triphenylphosphine/carbon tetrachloride or triphenylphosphine/diethyl azodicarboxylate optionally in the presence of a base such as potassium carbonate, N-ethyl-diisopropylamine or N,N-dimethylamino-pyridine conveniently at temperatures between 0 and 150° C., preferably at temperatures between 0 and 80° C., and the alkylation is carried out with a corresponding halide conveniently in a solvent such as methylene chloride, tetrahydrofuran, dioxane, dimethylsulphoxide, dimethylformamide or acetone optionally in the presence of a reaction accelerator such as sodium or potassium iodide and preferably in the presence of a base such as sodium carbonate or potassium carbonate or in the presence of a tertiary organic base such as N-ethyl-diisopropylamine or N-methyl-morpholine, which may simultaneously also serve as solvent, or optionally in the presence of silver carbonate or silver oxide at temperatures between −30 and 100° C., but preferably at temperatures between −10 and 80° C.

f. In order to prepare a compound of general formula I wherein R₂ contains a carboxy group:

converting a compound of general formula

R₁—A—B—R₂′″ (XI),

wherein

R₁, A and B are as hereinbefore defined and

R₂′″ has the meanings given for R₂ with the proviso that R₂ is substituted by a group which may be converted into a carboxy group, into a corresponding carboxy compound by hydrolysis, hydrogenolysis or thermolysis.

An example of a group which can be converted into a carboxy group is a carboxyl group protected by a protecting group, such as the functional derivatives thereof, e.g. the unsubstituted or substituted amides, esters, thioesters, trimethyl-silylesters, orthoesters or iminoesters thereof, the esters thereof with tertiary alcohols, e.g. the tert. butyl ester, and the esters thereof with aralkanols, e.g. the benzyl ester.

The hydrolysis is conveniently carried out either in the presence of an acid such as hydrochloric acid, sulphuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxane at temperatures between −10 and 120° C., e.g. at temperatures between ambient temperature and the boiling temperature of the reaction mixture.

The conversion of a tert. butyl or tert. butyloxycarbonyl group into a carboxy group can also be carried out by treating with an acid such as trifluoroacetic acid, formic acid, p-toluenesulphonic acid, sulphuric acid, hydrochloric acid, phosphoric acid or polyphosphoric acid, optionally in an inert solvent such as methylene chloride, chloroform, benzene, toluene, diethylether, tetrahydrofuran or dioxane, preferably at temperatures between −10 and 120° C., e.g. at temperatures between 0 and 60° C., or thermally, optionally in an inert solvent such as methylene chloride, chloroform, benzene, toluene, tetrahydrofuran or dioxane and preferably in the presence of a catalytic amount of an acid such as p-toluenesulphonic acid, sulphuric acid, phosphoric acid or polyphosphoric acid, preferably at the boiling temperature of the solvent used, e.g. at temperatures between 40 and 120° C.

The conversion of a benzyloxy or benzyloxycarbonyl group into a carboxy group may also be carried out hydrogenolytically in the presence of a hydrogenation catalyst such as palladium/charcoal in a suitable solvent such as methanol, ethanol, ethanol/water, glacial acetic acid, ethyl acetate, dioxane or dimethylformamide, preferably at temperatures between 0 and 50° C., e.g. at ambient temperature, and at a hydrogen pressure of 1 to 5 bar.

If according to the invention a compound of general formula I is obtained which contains an amino group, this can be converted by acylation into a correspondingly acylated compound of general formula I.

The subsequent acylation is conveniently carried out with a corresponding halide or anhydride in a solvent such as methylene chloride, chloroform, carbon tetrachloride, ether, tetrahydrofuran, dioxane, benzene, toluene, acetonitrile or sulpholane optionally in the presence of an inorganic or organic base such as triethylamine, N-ethyl-diisopropylamine, N-methyl-morpholine or pyridine at temperatures between −20 and 200° C., but preferably at temperatures between −10 and 160° C.

The subsequent acylation may however also be carried out with the free acid optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a trimethylsilyl, acetyl, benzoyl, methyl, ethyl, tert.butyl, trityl, benzyl or tetrahydropyranyl group,

protecting groups for a carboxy group may be a trimethylsilyl, methyl, ethyl, tert.butyl, benzyl or tetrahydropyranyl group and

protecting groups for an amino, alkylamino or imino group may be a formyl, acetyl, trifluoroacetyl, ethoxycarbonyl, tert.butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. in the presence of iodotrimethylsilane, at temperatures between 0 and 120° C., preferably at temperatures between 10 and 100° C.

However, a benzyl, methoxybenzyl or benzyloxycarbonyl group is cleaved, for example hydrogenolytically, e.g. with hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisol.

A tert.butyl or tert.butyloxycarbonyl group is preferably cleaved by treating with an acid such as trifluoroacetic acid or hydrochloric acid or by treating with iodotrimethylsilane optionally using a solvent such as methylene chloride, dioxane, methanol or diethylether.

A trifluoroacetyl group is preferably cleaved by treating with an acid such as hydrochloric acid, optionally in the presence of a solvent such as acetic acid at temperatures between 50 and 120° C. or by treating with sodium hydroxide solution optionally in the presence of a solvent such as tetrahydrofuran at temperatures between 0 and 50° C.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

The compounds of general formulae II to XI used as starting materials are known from the literature in some cases or may be prepared by methods known from the literature, as described in the Examples.

Moreover, the compounds of general formula I obtained may be resolved into their enantiomers and/or diastereomers, as mentioned hereinbefore. Thus, for example, compounds with at least one optically active carbon atom may be separated into their enantiomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 stereogenic centres may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

Furthermore, the compounds of formula I obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain an acidic group such as a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine and triethanolamine.

As already mentioned hereinbefore, the carboxylic acid amides of general formula I and the salts thereof, particularly the physiologically acceptable salts thereof, have an inhibiting effect on telomerase.

The inhibiting effect of the carboxylic acid amides of general formula I on telomerase was investigated as follows:

Materials and Methods:

1. Preparation of nuclear extracts from HeLa cells: Nuclear extracts were prepared according to Dignam (Dignam et al. in Nucleic Acids Res. 11, 1475-1489 (1983)). All the steps were carried out at 4° C., all the equipment and solutions were pre-cooled to 4° C. At least 1×10⁹ HeLa-S3 cells growing in suspension culture (ATCC catalogue number CCL-2.2) were harvested by centrifuging for 5 minutes at 1000×g and washed once with PBS buffer (140 mM KCl; 2.7 mM KCl; 8.1 mM Na₂HPO₄; 1.5 mM KH₂PO₄) . After the cell volume had been determined, the cells were suspended in 5 times the volume of hypotonic buffer (10 mM HEPES/KOH, pH 7.8; 10 mM KCl; 1.5 mM MgCl₂) and then left for 10 minutes at 4° C. After centrifuging for 5 minutes at 1000×g the cell pellet was suspended in twice the volume of hypotonic buffer in the presence of 1 mM DTE and 1 mM PMSF and broken up with a Dounce homogeniser. The homogenised material was made isotonic with 0.1 volume of 10-fold saline buffer (300 mM HEPES/KOH, pH 7.8; 1.4 M KCl; 30 mM MgCl₂). The cell nuclei were separated from the ingredients of the cytoplasm by centrifuging and then suspended in twice the volume of nuclear extraction buffer (20 mM HEPES/KOH, pH 7.9; 420 mM KCl; 1.5 mM MgCl₂; 0.2 mM EDTA; 0.5 mM DTE; 25% glycerol). The nuclei were broken up using a Dounce homogeniser and incubated for 30 minutes at 4° C. with gentle stirring. Any insoluble ingredients were removed by centrifuging for 30 minutes at 10.000 rpm (SS-34 Rotor). Then the nuclear extract was dialysed for 4-5 hours against AM-100 buffer (20 mM tris/HCl, pH 7.9; 100 mM KCl; 0.1 mM EDTA; 0.5 mM DTE; 20% glycerol). The nuclear extracts obtained were frozen in liquid nitrogen and stored at −80° C.

2. Telomerase test: The activity of telomerase in nuclear extracts from HeLa cells was determined using the method described by Morin (Morin in Cell 59. 521-529 (1989)). The nuclear extract (up to 20 μl per reaction) was incubated for 120 minutes at 30° C. in a volume of 40 μl in the presence of 25 mM Tris/HCl pH 8.2, 1.25 mM DATP, 1.25 mM TTP, 6.35 μM dGTP; 15 μCi α-³²P-dGTP (3000 Ci/mmol), 1 mM MgCl₂, 1 mM EGTA, 1.25 mM spermidine, 0.25 U RNasin, and 2.5 μM of an oligonucleotide primer (for example TEA-fw [CAT ACT GGC GAG CAG AGT T], or TTA GGG TTA GGG TTA GGG) (=telomerase reaction). If the inhibition constant of potential telomerase inhibitors was to be determined, these were also added to the telomerase reaction in a concentration range of from 1 nM to 100 μM. The reaction was then stopped by the addition of 50 μl of RNase stop buffer (10 mM tris/HCL, pH 8.0; 20 mM EDTA; 0.1 mg/ml of RNase A 100 U/ml of RNase T1; 1000 cpm of an α-³²P-dGTP labelled, 430 bp DNA fragment) and incubation was continued for a further 15 minutes at 37° C. Proteins present in the reaction mixture were cleaved by the addition of 50 μl of proteinase K buffer (10 mM tris/HCL, pH 8.0; 0.5% SDS; 0.3 mg/ml of proteinase K) and subsequent incubation for 15 min at 37° C. The DNA was purified by extracting twice with phenol-chloroform and precipitated by adding 2.4 M ammonium acetate; 3 μg tRNA and 750 μl ethanol. Then the precipitated DNA was washed with 500 μl of 70% ethanol, dried at ambient temperature, taken up in 4 μl of formamide probe buffer (80% (V/V) formamide; 50 mM of tris-borate, pH 8.3; 1 mM EDTA; 0.1 (w/v) of xylene cyanol; 0.1% (w/v) bromophenol blue) and separated by electrophoresis on a sequence gel (8% polyacrylamide, 7 M urea, 1×TBE buffer). The DNA synthesised by telomerase in the presence or absence of potential inhibitors was identified and quantified by Phospho-Imager Analysis (Molecular Dynamics) and in this way the concentration of inhibitor which inhibits the telomerase activity by 50% (IC₅₀) was determined. The radiolabelled DNA fragment to which the RNase stop buffer had been added was used as an internal control for the yield.

The following Table gives the IC₅₀ values of some inhibitors by way of example:

| Example No. | IC₅₀ [μM] |
|---|---|
| 3 | <5 |
| 35 | <1 |
| 50 | <1 |

The following abbreviations were used in the foregoing description:

| | |
|---|---|
| bp | base pairs |
| DNA | deoxyribonucleic acid |
| DTE | 1,4-dithioerythritol |
| dATP | deoxyadenosine triphosphate |
| dGTP | deoxyguanosine triphosphate |
| EDTA | ethylendiamine-tetraacetic acid |
| EGTA | ethyleneglycol-bis-(2-aminoethyl)-tetraacetic acid |
| HEPES | 4-(2-hydroxyethyl)-piperazine-1-ethanesulphonic |
| | acid |
| PMSF | phenylmethanesulphonylfluoride |
| RNase | ribonuclease |
| RNasin ® | ribonuclease inhibitor (Promega GmbH, Mannheim) |
| tRNA | transfer ribonucleic acid |
| TTP | thymidine triphosphate |
| TRIS | tris-(hydroxymethyl)-aminomethane |
| TBE | TRIS-borate-EDTA |
| rpm | revolutions per minute |

In view of their biological properties, the carboxylic acid amides of general formula I are suitable for treating patho-physiological processes which are characterised by an increased telomerase activity. These are e.g. tumour diseases such as carcinomas, sarcomas and leukaemias including skin cancer (e.g. plate epithelial carcinoma, basalioma, melanoma), small-cell bronchial carcinoma, non-small-cell bronchial carcinoma, salivary gland carcinoma, oesophageal carcinoma, laryngeal carcinoma, pharyngeal carcinoma, thyroid carcinoma, gastric carcinoma, colorectal carcinoma, pancreatic carcinoma, carcinoma of the liver, carcinoma of the breast, uterine carcinoma, vaginal carcinoma, ovarian carcinoma, prostate carcinoma, testicular carcinoma, bladder carcinoma, renal carcinoma, Wilms' tumour, retinoblastoma, astrocytoma, oligodendroglioma, meningioma, neuroblastoma, myeloma, medulloblastoma, neurofibrosarcoma, thymoma, osteosarcoma, chondrosarcoma, Ewing's sarcoma, fibrosarcoma, histiocytoma, dermatofibrosarcoma, synovialoma, leiomyosarcoma, rhabdomyosarcoma, liposarcoma, Hodgkin's lymphoma, non-Hodgkin's lymphoma, chronic myeloid leukaemia, chronic lymphatic leukaemia, acute promyelocytic leukaemia, acute lymphoblastoid leukaemia and acute myeloid leukaemia.

In addition, the compounds may also be used to treat other diseases which have an increased rate of cell division or increased telomerase activity, such as e.g. epidermal hyperproliferation (psoriasis), inflammatory processes (rheumatoid arthritis), diseases of the immune system, etc.

The compounds are also useful for treating parasitic diseases in man and animals, such as e.g. worm or fungal diseases as well as diseases caused by protozoan pathogens, such as e.g. Zooflagellata (Trypanosoma, Leishmania, Giardia), Rhizopoda (Entamoeba spp.), Sporozoa (Plasmodium spp., Toxoplasma spp.), Ciliata, etc.

For this purpose the carboxylic acid amides of general formula I may optionally be used in conjunction with other pharmacologically active compounds and therapeutic preparations which will reduce tumour size and incorporated in conventional galenic preparations. These may be used, for example, in tumour therapy, in monotherapy or in conjunction with irradiation, surgical interventions or other anti-tumour therapeutics, e.g. in conjunction with topoisomerase inhibitors (e.g. etoposide), mitosis inhibitors (e.g. paclitaxel, vinblastin), cell cycle inhibitors (e.g. flavopyridol), inhibitors of signal transduction (e.g. farnesyltransferase inhibitors), compounds which interact with nucleic acid (e.g. cis-platin, cyclophosphamide, adriamycin), hormone antagonists (e.g. tamoxifen), inhibitors of metabolic processes (e.g. 5-FU etc.), cytokines (e.g. interferons), tumour vaccines, antibodies, etc. These combinations may be given either simultaneously or sequentially.

The daily dose is 20 to 600 mg by oral or intravenous route, divided up into one to four doses a day. For this purpose the compounds of general formula I, optionally in conjunction with the other active substances mentioned above, may be formulated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, cetylstearyl alcohol, carboxymethylcellulose or fatty substances such as hard fat or suitable mixtures thereof to produce conventional galenic preparations such as plain or coated tablets, capsules, powders, suspensions or suppositories.

The following Examples are intended to illustrate the invention in more detail:

### EXAMPLE 1

### 2-[4-(2-oxo-2H-chromen-3-yl)-thiazol-2-ylaminocarbonyl]-benzoic acid

a. 3-acetyl-2-chroman-2-one

A mixture of 2 g (16.4 mmol) of salicylaldehyde and 2.1 g (16.4 mmol) of acetoacetate is combined with 0.1 g piperidine at 0° C. and stirred at ambient temperature until the mixture sets in a solid mass. Then it is triturated with ethanol, filtered and the residue recrystallised from water.

Yield: 2.2 g (70% of theory),

C₁₁H₈O₃ (188.18)

Mass spectrum:

M⁺=188

b. 3-(2-bromoacetyl)-chroman-2-one

A solution of 2.2 g (11.4 mmol) of 3-acetyl-2-chroman-2-one in 10 ml chloroform is slowly combined with 0.6 ml (11.4 mmol) of bromine in 2 ml chloroform and then heated over a water bath for 30 minutes. It is then cooled in an ice bath, the product is suction filtered and dried.

Yield: 2.4 g (79% of theory),

R_{f} value: 0.48 (silica gel; ethyl acetate/cyclohexane=1:2)

C₁₁H₇BrO₃ (267.08)

Mass spectrum:

M⁺=266/8 (bromine isotope)

c. 2-amino-4-(2-oxo-2H-chroman-3-yl)-thiazole

To a solution of 0.7 g (8.9 mmol) of thiourea in 20 ml of ethanol are added 2.4 g (8.9 mmol) of 3-(2-bromoacetyl)-chroman-2-one. Then the mixture is refluxed for 15 minutes. The reaction mixture is cooled, diluted with water and made alkaline with ammonia solution. The precipitate formed is suction filtered.

Yield: 2.2 g (99% of theory),

R_{f} value: 0.21 (silica gel; ethyl acetate/cyclohexane=1:2)

C₁₂H₈N₂O₂S (244.27)

Mass spectrum:

M⁺=244

d. 2-[4-(2-oxo-2H-chromen-3-yl)-thiazol-2-ylaminocarbonyl]-benzoic acid

0.1 g (0.4 mmol) of 2-amino-4-(2-oxo-2H-chroman-3-yl)-thiazole and 0.2 9 (1.6 mmol) of phthalic anhydride are stirred in 2 ml of pyridine for 3.5 days. Then water is added and the mixture is concentrated by evaporation in vacuo. The residue is suspended in acetone/water (3:1), the crude product is suction filtered and recrystallised from acetone.

Yield: 83 mg (52% of theory),

C₂₀H₁₂N₂O₅S (392.39)

Mass spectrum:

(M+H)⁺=393

(M−H)⁻=391

(M−H₂O)⁻=374

### EXAMPLE 2

### 2-(4-phenyl-thiazol-2-ylaminocarbonyl)-benzoic acid

Prepared analogously to Example 1d from 2-amino-4-phenyl-thiazole and phthalic anhydride in pyridine.

Yield: 55% of theory,

C₁₇H₁₂N₂O₃S (324.36)

Mass spectrum:

(M+H)⁺=325

(M−H)⁻=323

M⁺=324

### EXAMPLE 3

### 2-[4-(naphthalin-2-yl)-thiazol-2-ylaminocarbonyl]-benzoic acid

Prepared analogously to Example 1d from 2-amino-4-(naphthalin-2-yl)-thiazole and phthalic anhydride in pyridine.

Yield: 30% of theory,

C₂₁H₁₄N₂O₃S (374.42)

Mass spectrum:

(M+H)⁺=375

(M−H)⁻=373

### EXAMPLE 4

### 2-(4-styryl-thiazol-2-ylaminocarbonyl)-benzoic acid

a. 1-bromo-4-phenyl-but-3-en-2-one

5.0 g (34.2 mmol) of benzylidene acetone are placed in 200 ml of tetrahydrofuran, combined batchwise with 20.1 g (34.9 mmol) of triphenylphosphinepropionic acid bromide and stirred for 1 hour at ambient temperature. The solvent is distilled off and the residue is chromatographed on silica gel, eluting with cyclohexane/ethyl acetate (7:1).

Yield: 4.6 g (59% of theory),

R_{f} value: 0.65 (silica gel; ethyl acetate/cyclohexane=1:4)

C₁₀H₉BrO (225.09)

Mass spectrum: M⁺=224/26 (bromine isotope)

b. 2-amino-4-styryl-thiazole

Prepared analogously to Example 1c from 1-bromo-4-phenyl-but-3-en-2-one and thiourea in ethanol.

Yield: 47% of theory,

R_{f} value: 0.14 (silica gel; ethyl acetate/cyclohexane=1:4)

C₁₁H₁₀N₂S (202.28)

Mass spectrum:

M⁺=202

(M+H)⁺=203

c. 2-(4-Styryl-thiazol-2-ylaminocarbonyl)-benzoic acid

Prepared analogously to Example 1d from 2-amino-4-styryl-thiazole and phthalic anhydride in pyridine.

Yield: 63% of theory,

C₁₉H₁₄N₂O₃S (350.40)

Mass spectrum:

M⁺=350

(M−H)⁻=349

### EXAMPLE 5

### 3,6-dichloro-2-[4-(naphthalin-2-yl)-thiazol-2-ylaminocarbonyl]-benzoic acid

Prepared analogously to Example 1d from 2-amino-4-(naphthalin-2-yl)-thiazole and 3,6-dichloro-phthalic anhydride in pyridine.

Yield: 69% of theory,

C₂₁H₁₂Cl₂N₂O₃S (443.31)

Mass spectrum:

(M−H)⁻=441/3/5 (chlorine isotope)

### EXAMPLE 6

### 4,5-dimethoxy-2-[4-(naphthalin-2-yl)-thiazol-2-ylaminocarbo-nyl]-benzoic acid

Prepared analogously to Example 1d from 2-amino-4-(naphthalin-2-yl)-thiazole and 4,5-dimethoxy-phthalic anhydride in pyridine.

Yield: 86% of theory,

C₂₃H₁₈N₂O₅S (434.47)

Mass spectrum:

(M−H)⁻=433

(M−H₂O)⁻=416

### EXAMPLE 7

### 2-[4-(naphthalin-1-yl)-thiazol-2-ylaminocarbonyl]-benzoic acid

Prepared analogously to Example 1d from 2-amino-4-(naphthalin-1-yl)-thiazole and phthalic anhydride in pyridine.

Yield: 51% of theory,

C₂₁H₁₄N₂O₃S (374.42)

Mass spectrum:

(M+H)⁺=375

(M−H)⁻=373

### EXAMPLE 8

### 2-[4-(6-methyl-naphthalin-2-yl)-thiazol-2-ylaminocarbonyl]-benzoic acid

Prepared analogously to Example 1d from 2-amino-4-(6-methyl-naphthalin-2-yl)-thiazole and phthalic anhydride in pyridine.

Yield: 45% of theory,

C₂₂H₁₆N₂O₃S (388.45)

Mass spectrum:

(M−H)⁻=387

(M−H₂O)⁻=370

### EXAMPLE 9

### 2-[4-(naphthalin-2-yl)-thiazol-2-ylcarbonylamino]-benzoic acid

a. ethyl 4-(naphthalin-2-yl)-thiazol-2-ylcarboxylate 0.7 9 (2.78 mmol) of 2-bromo-1-naphthalin-2-yl-ethanone are placed in 7 ml of ethanol, combined with 0.4 g (3.06 mmol) of ethyl thiooxamidate and refluxed for 1 hour. After cooling to ambient temperature the mixture is combined with water and suction filtered.

Yield: 0.7 g (89% of theory),

R_{f} value: 0.55 (silica gel; ethyl acetate/cyclohexane=1:4)

C₁₆H₁₃NO₂S (283.35)

Mass spectrum:

M⁺=283

b. 4-(naphthalin-2-yl)-thiazole-2-carboxylic acid

1.1 g (3.8 mmol) of ethyl 4-(naphthalin-2-yl)-thiazole-2-carboxylate are placed in a mixture of 10 ml of water and 8 ml of tetrahydrofuran and after the addition of 0.8 g (19.4 mmol) of lithium hydroxide stirred for 1.5 hours. Then the mixture is acidified with lN hydrochloric acid, the organic solvent is distilled off and the precipitate is suction filtered.

Yield: 0.6 g (58% of theory),

R_{f} value: 0.10 (silica gel; dichloromethane/methanol/ammonia=9:1:0.1)

C₁₄H₉NO₂S (255.30)

Mass spectrum:

M⁺=255

c. 4-(naphthalin-2-yl)-thiazole-2-carboxylic acid chloride

0.6 g (2.2 mmol) of 4-(naphthalin-2-yl)-thiazole-2-carboxylic acid are suspended in 4 ml of thionyl chloride and after the addition of one drop of dimethylformamide refluxed for 45 minutes. The solvent is distilled off and the oil obtained is reacted without any further purification.

Yield: 0.6 g (100% of theory).

d. 2-[4-(naphthalin-2-yl)-thiazol-2-ylcarbonylamino]-benzoic acid

To a solution of 0.3 g (2.2 mmol) of anthranilic acid in 20 ml of tetrahydrofuran and 0.5 ml (3.2 mmol) of triethylamine is added dropwise a solution of 0.6 g (2.2 mmol) of 4-(naphthalin-2-yl)-thiazole-2-carboxylic acid chloride in 15 ml of tetrahydrofuran. The reaction mixture is stirred for 2 hours. Then it is concentrated by evaporation, the residue is suspended in 1N hydrochloric acid and suction filtered.

Yield: 0.6 g (75% of theory),

C₂₁H₁₄N₂O₃S (374.42)

Mass spectrum:

M⁺=374

(M−H₂O)⁻=356

### EXAMPLE 10

### 2-(4-Styryl-thiazol-2-ylcarbonylamino)-benzoic acid

Prepared analogously to Example 9d from anthranilic acid and 4-styryl-thiazole-2-carboxylic acid chloride in tetrahydrofuran.

Yield: 59% of theory,

C₁₉H₁₄N₂O₃S (350.40)

Mass spectrum:

M⁺=350

(M−H₂O)⁻=332

### EXAMPLE 11

### 2-[4-(naphthalin-1-yl)--thiazol-2-ylcarbonylamino]-benzoic acid

Prepared analogously to Example 9d from anthranilic acid and 4-(naphthalin-1-yl)-thiazole-2-carboxylic acid chloride. The crude product obtained is then purified by chromatography on silica gel (methylene chloride/methanol/conc. ammonia=9.:1:0.1).

Yield: 14% of theory,

C₂₁H₁₄N₂O₃S (374.42)

Mass spectrum:

M⁺=374

(M−H)⁻=373

### EXAMPLE 12

### 2-[4-(3,4-dimethoxy-phenyl)-thiazol-2-ylaminocarbonyl]-benzoic acid amide

Prepared analogously to Example 1d from 2-amino-4-(3,4-dimethoxy-phenyl)-thiazole and phthalic anhydride in pyridine. The crude product obtained is then purified by chromatography on silica gel (methylene chloride/methanol/conc.ammonia =9:1:0.1).

Yield: 16% of theory,

C₁₉H₁₇N₃O₄S (383.43)

Mass spectrum:

(M+H)⁺=384

### EXAMPLE 13

### 2-[4-(3,4-dimethoxy-phenyl)-thiazol-2-ylaminocarbonyl]-benzoic acid

Prepared analogously to Example 1d from 2-amino-4-(3,4-dimethoxy-phenyl)-thiazole and phthalic anhydride in pyridine.

Yield: 31% of theory,

C₁₉H₁₆N₂O₅S (384.41)

Mass spectrum:

(M−H)⁻=383

### EXAMPLE 14

### 2-[4-(5,6,7,8-tetrahydro-naphthalin-2-yl)-thiazol-2-ylamino-carbonyl]-benzoic acid

Prepared analogously to Example 1d from 2-amino-4-(5,6,7,8-tetrahydro-naphthalin-2-yl)-thiazole and phthalic anhydride in pyridine.

Yield: 64% of theory,

C₂₁H₁₈N₂O₃S (378.45)

Mass spectrum:

(M−H)⁻=377

### EXAMPLE 15

### 2-[4-(2,3-dihydro-benzo[1,4]dioxin-6-yl)-thiazol-2-ylamino-carbonyl]-benzoic acid

Prepared analogously to Example 1d from 2-amino-4-(2,3-dihydro-benzo[1,4]dioxin-6-yl)-thiazole and phthalic anhydride in pyridine.

Yield: 38% of theory,

C₁₉H₁₄N₂O₅S (382.40)

Mass spectrum:

(M−H)⁻=381

### EXAMPLE 16

### 2-[4-(benzo[1,3]dioxol-5-yl)-thiazol-2-ylaminocarbonyl]-benzoic acid

Prepared analogously to Example 1d from 2-amino-4-benzo[1,3]dioxol-5-yl-thiazole and phthalic anhydride in pyridine.

Yield: 71% of theory,

C₁₈H₁₂N₂O₅S (368.37)

Mass spectrum:

M⁺=368

### EXAMPLE 17

### 2-[4-(3,4-dimethyl-phenyl)-thiazol-2-ylaminocarbonyl]-benzoic acid

Prepared analogously to Example 1d from 2-amino-4-(3,4-dimethyl-phenyl)-thiazole and phthalic anhydride in pyridine.

Yield: 78% of theory,

C₁₉H₁₆N₂O₃S (352.41)

Mass spectrum:

M⁺=352

### EXAMPLE 18

### 2-[5-(naphthalin-1-yl)-thiophen-2-ylcarbonylamino]-benzoic acid

a. 5-(naphthalin-1-yl)-thiophene-2-carboxaldehyde

1.2 ml (10.4 mmol) of 5-bromothiophene-2-carboxaldehyde and 0.4 g (0.31 mmol) of tetrakis-triphenylphosphine-palladium are stirred in 20 ml of dimethoxyethane for 15 minutes. Then a solution of 2.2 g (12.5 mmol) of 1-naphthylboric acid in 4 ml of ethanol and 11 ml of 2N sodium carbonate is added. The reaction mixture is refluxed for 5 hours. After cooling the mixture is diluted with diethyl ether, the organic phase is separated off and concentrated by evaporation. The residue is purified by chromatography, eluting with cyclohexane/ethyl acetate (95:5).

Yield: 2.4 g (95% of theory),

R_{f} value: 0.09 (silica gel; cyclohexane)

C₁₅H₁₀OS (238.31)

Mass spectrum:

M⁺=238

b. 5-(naphthalin-1-yl)-thiophene-2-carboxylic acid

2.3 g (9.6 mmol) of 5-(naphthalin-1-yl)-thiophen-2-carboxaldehyde are placed in 40 ml of ethanol and after the addition of 8.2 g (48.2 mmol) of silver nitrate in 6 ml of water and 2.7 g (48.2 mmol) of potassium hydroxide the mixture is stirred in 40 ml of water for 1 hour. Then the precipitate is filtered off, the aqueous phase is separated off, adjusted to pH 4 with hydrochloric acid and extracted with ethyl acetate. The combined organic extracts are dried and concentrated by evaporation.

Yield: 1.5 g (61% of theory),

R_{f} value: 0.17 (silica gel; cyclohexane/ethyl acetate=1:2)

C₁₅H₁₀O₂S (254.31)

Mass spectrum:

M⁺=254

c. 5-(naphthalin-1-yl)-thiophen-2-ylcarboxylic acid chloride

Prepared analogously to Example 9c from 5-(naphthalin-1-yl)-thiophen-2-carboxylic acid and thionyl chloride with the addition of dimethylformamide.

Yield: 100% of theory.

d. 2-[5-(naphthalin-1-yl)-thiophen-2-ylcarbonylamino]-benzoic acid

Prepared analogously to Example 9d from 5-(naphthalin-1-yl-thiophen)-2-carboxylic acid chloride and anthranilic acid in tetrahydrofuran.

Yield: 53% of theory,

C₂₂H₁₅NO₃S (373.43)

Mass spectrum:

M⁺=373

(M−H)⁻=372

(M−H₂O)⁻=355

### EXAMPLE 19

### 2-[5-(naphthalin-2-yl)-thiophen-2-ylcarbonylamino]-benzoic acid

Prepared analogously to Example 9d from 5-naphthalin-2-yl-thiophen-2-carboxylic acid chloride and anthranilic acid in tetrahydrofuran.

Yield: 13% of theory,

C₂₂H₁₅NO₃S (373.43)

Mass spectrum:

M⁺=373

(M−H₂O)⁻=355

### EXAMPLE 20

### 2-[5-(naphthalin-2-yl)-furan-2-ylcarbonylamino]-benzoic acid

a. methyl 5-bromofuran-2-carboxylate

A solution of 3.0 g (15.7 mmol) of 5-bromofuran-2-carboxylic acid, 1.9 ml (47.1 mmol) of methanol and 0.5 ml of conc. sulphuric acid in 10 ml of dichloroethane is refluxed for 20 hours. Then the reaction mixture is added to water, the organic phase is separated off, washed with saturated sodium hydrogen carbonate solution, dried and concentrated by evaporation.

Yield: 3.0 g (93% of theory),

R_{f} value: 0.71 (silica gel; cyclohexane/ethyl acetate=2:1)

C₆H₅BrO₃ (205.01)

Mass spectrum:

(M+Na)⁺=227/29 (bromine isotope)

b. 5-(naphthalin-2-yl)-furan-2-carboxylic acid

3.0 g (14.5 mmol) of methyl 5-bromofuran-2-carboxylate and 0.5 g (0.43 mmol) of tetrakis-triphenylphosphine-palladium are stirred in 30 ml of toluene for 15 minutes. Then 2.7 g (15.9 mmol) of 2-naphthylboric acid in 7 ml of ethanol and 14.5 ml of 2N sodium carbonate are added. The reaction mixture is refluxed for 4.5 hours. After cooling it is acidified with 1N hydrochloric acid, diluted with ethyl acetate, the organic phase is separated off, dried and concentrated by evaporation. The crude product is dissolved in 42 ml of tetrahydrofuran, diluted with 55 ml of water and after the addition of 3.0 g (72.5 mmol) of lithium hydroxide stirred for 5 hours. Then it is acidified with hydrochloric acid, the tetrahydrofuran is distilled off and the precipitate is suction filtered.

Yield: 3.0 g (88% of theory),

R_{f} value: 0.24 (silica gel; dichloromethane/methanol=9:1)

C₁₅H₁₀₃ (238.25)

Mass spectrum:

(M+H)⁺=239

(M−H)⁻=237

c. 2-[5-(naphthalin-2-yl)-furan-2-ylcarbonylamino]-benzoic acid

Prepared analogously to Example 9c from 5-(naphthalin-2-yl)-furan-2-carboxylic acid and thionyl chloride and subsequent reaction analogous to Example 9d with anthranilic acid in tetrahydrofuran.

Yield: 50% of theory,

C₂₂H₁₅NO₄ (357.37)

Mass spectrum:

M⁺=357

(M−H₂O)⁻=339

(M+H)⁺=358

(M−H)⁻=356

### EXAMPLE 21

### 2-[4-(naphthalin-2-yl)-1H-imidazol-2-ylaminocarbonyl]-benzoic acid

a. N-[4-(naphthalin-2-yl)-1H-imidazol-2-yl]-acetamide

A mixture of 2.4 g (24.1 mmol) of 1-acetyl-guanidine and 2.0 g (8 mmol) of 2-bromo-1-(naphthalin-2-yl)-ethanol is stirred in 28 ml of dimethylformamide for 23 hours at ambient temperature. Then the solvent is distilled off, the residue is washed with water and filtered off. The crude product is recrystallised from ethanol.

Yield: 0.9 g (47% of theory),

R_{f} value: 0.41 (silica gel; dichloromethane/methanol=9.5:0.5)

C₁₅H₁₃N₃O (251.29)

Mass spectrum:

M⁺=251

b. 2-amino-4-(naphthalin-2-yl)-1H-imidazole

0.4 g (1.6 mmol) of N-[4-(naphthalin-2-yl)-1H-imidazol-2-yl]-acetamide are suspended in 10 ml of water and 10 ml of methanol and after the addition of 0.2 ml of conc. sulphuric acid refluxed for 11 hours. Then the pH is adjusted to 10 with 1% methanolic potassium hydroxide solution and the mixture is concentrated by evaporation. The crude product is purified by chromatography, eluting with dichloromethane/methanol/ammonia (10:1:0.1).

Yield: 0.2 g (51% of theory),

R_{f} value: 0.37 (silica gel; dichloromethane/methanol/ammonia=9:1:0.1)

C₁₃H₁₁N₃ (209.25)

Mass spectrum:

(M+H)⁺=210

(M−H)⁻=208

c. 2-[4-(naphthalin-2-yl)-1H-imidazol-2-ylaminocarbonyl]-benzoic acid

Prepared analogously to Example 1d from 2-amino-4-(naphthalin-2-yl)-1H-imidazole and phthalic anhydride in pyridine.

Yield: 31% of theory,

C₂₁H₁₅N₃O₃ (357.37)

Mass spectrum:

(M−H₂O)⁻=339

(M−H)⁻=356

### EXAMPLE 22

### 2-[l-methyl-4-(naphthalin-2-yl)-1H-imidazol-2-yl-aminocarbonyl]-benzoic acid

a. N-[1-methyl-4-(naphthalin-2-yl)-1H-imidazol-2-yl]-acetamide

0.5 g (2 mmol) of N-[4-(naphthalin-2-yl)-1H-imidazol-2-yl]-acetamide, 0.1 ml (2 mmol) of methyl iodide and 0.1 g (1 mmol) of potassium carbonate are refluxed in 20 ml acetone for 4 hours. Then a further 0.2 ml (4 mmol) of methyl iodide are added and refluxing is continued for another 9 hours. The precipitate is filtered off, the mother liquor concentrated by evaporation and the residue purified by chromatography, eluting with dichloromethane/methanol (98:2).

Yield: 0.1 g (24% of theory),

R_{f} value: 0.34 (silica gel; dichloromethane/methanol 9.5:0.5)

C₁₆H₁₅N₃O (265.32)

Mass spectrum:

(M+H)⁺=266

(M−H)⁻=264

b. 2-[l-methyl-4-(naphthalin-2-yl)-1H-imidazol-2-yl-aminocarbonyl]-benzoic acid

Prepared analogously to Example 21b from N-{1-methyl-4-(naphthalin-2-yl)-1H-imidazol-2-yl]-acetamide and conc. sulphuric acid in methanol/water and subsequent reaction analogous to Example 1d with phthalic anhydride in pyridine.

Yield: 18% of theory,

C₂₂H₁₇N₃O₃ (371.40)

Mass spectrum:

(M−H)⁻=370

(M−H₂O)⁻=353

### EXAMPLE 23

### 2-[5-(naphthalin-2-yl)-[1,3,4]thiadiazol-2-ylaminocarbonyl]-benzoic acid

a. 2-amino-5-(naphthalin-2-yl)-[1,3,4]thiadiazole

20 g of polyphosphoric acid are heated to 80 to 90° C., combined with a mixture of 2 g (11.6 mmol) of 2-naphthylcarboxylic acid and 1.1 g (11.6 mmol) of thiosemicarbazide within 30 minutes and then stirred for 4 hours at 90° C. The mixture is then cooled, poured onto ice water and made alkaline with ammonia. The crude product is suction filtered and purified by chromatography, eluting with dichloromethane/methanol (99:1 to 95:5).

Yield: 1.5 g (56% of theory),

R_{f} value: 0.31 (silica gel; dichloromethane/methanol=9.5:0.5)

C₁₂H₉N₃S (227.29)

Mass spectrum:

M⁺=227

b. 2-[5-(naphthalin-2-yl)-[1,3,4]thiadiazol-2-ylaminocarbonyl]-benzoic acid

Prepared analogously to Example 1d from 2-amino-5-(naphthalin-2-yl)-[1,3,4]thiadiazole and phthalic anhydride in pyridine.

Yield: 26% of theory,

C₂₀H₁₃N₃O₃S (375.41)

Mass spectrum:

(M−H)⁻=374

(M−H₂O)⁻=357

### EXAMPLE 24

### 2-[4-(3,4-dimethoxy-phenyl)-thiazol-2-yl-N-methyl-aminocarbonyl]-benzoic acid

Prepared analogously to Example 1c from 2-bromo-1-(3,4-dimethoxy-phenyl)-ethanone and N-methyl-thiourea in ethanol and subsequent reaction analogous to Example 1d with phthalic anhydride in pyridine.

Yield: 63% of theory,

R_{f} value:,0.58 (silica gel; dichloromethane/methanol=9:1)

C₂₀H₁₈N₂O₅S (398.44)

Mass spectrum:

M⁺=398

(M−H)⁻=397

### EXAMPLE 25

### 2-[4-(3-methoxy-naphthalin-2-yl)-thiazol-2-ylaminocarbonyl]-benzoic acid

a. 3-methoxy-naphthaline-2-carboxylic acid chloride

Prepared analogously to Example 9c from 3-methoxy-naphthaline-2-carboxylic acid and thionyl chloride.

Yield: 5.5 g (100% of theory).

b. diethyl 2-(3-methoxy-naphthaline-2-carbonyl)-malonate

0.7 g (30 mmol) of magnesium are placed in 0.7 ml of ethanol and heated until the reaction mixture boils. Then 20 ml ether and 4.5 ml (30 mmol) of diethyl malonate with 1.7 ml (30 mmol) of ethanol in 10 ml ether are added dropwise. After the addition has ended the mixture is refluxed for a further 3 hours. After stirring overnight, 5.5 g (25 mmol) of 3-methoxy-naphthalin-2-carboxylic acid chloride in 50 ml ether are added dropwise, then the mixture is refluxed for 1 hour. It is then cooled, 6 g of conc. sulphuric acid in 50 ml of water and 80 ml of ether are added and the mixture is stirred for 2 hours at ambient temperature. The ethereal phase is separated off, dried and concentrated by evaporation.

Yield: 10.3 g (99% of theory),

R_{f} value: 0.78 (silica gel; dichloromethane/methanol=9:1)

c. 1-(3-methoxy-naphthalin-2-yl)-ethanone

A mixture of 8.6 g (25 mmol) of diethyl 2-(3-methoxy-naphthalin-2-carbonyl)-malonate, 15 ml of glacial acetic acid, 2 ml of conc. sulphuric acid and 10 ml of water is refluxed for 4 hours. Then it is poured onto ice water and made alkaline with 20% sodium hydroxide solution. After extraction with ether it is dried and concentrated by evaporation.

Yield: 3.3 g (65% of theory),

R_{f} value: 0.6 (silica gel; cyclohexane/ethyl acetate=2:1)

d. 2-amino-4-(3-methoxy-naphthalin-2-yl)-thiazole

Prepared analogously to Example 1b from 1-(3-methoxy-naphthalin-2-yl)-ethanone and bromine in dichloromethane and subsequent reaction analogous to Example 1c with thiourea in ethanol.

Yield: 80% of theory,

R_{f} value: 0.65 (silica gel; dichloromethane/methanol=9:1)

C₁₄H₁₂N₂OS (256.33)

Mass spectrum:

(M+H)⁺=257

e. 2-[4-(3-methoxy-naphthalin-2-yl)-thiazol-2-ylaminocarbonyl]-benzoic acid

Prepared analogously to Example 1d from 2-amino-4-(3-methoxy-naphthalin-2-yl)-thiazole and phthalic anhydride in pyridine.

Yield: 20% of theory, C₂₂H₁₆N₂O₄S (404.45)

Mass spectrum:

(M−H)⁻=403

### EXAMPLE 26

### 2-[4-(naphthalin-2-yl)-thiazol-2-yl-N-methyl-aminocarbonyl]-benzoic acid

Prepared analogously to Example 1d from 2-methylamino-4-(naphthalin-2-yl)-thiazole and phthalic anhydride in pyridine.

Yield: 86% of theory,

C₂₂H₁₆N₂O₃S (388.45)

Mass spectrum:

M⁺=388

### EXAMPLE 27

### 2-[4-(2-oxo-1,2-dihydro-quinolin-3-yl)-thiazol-2-ylaminocarbonyl]-benzoic acid

a. 2-amino-benzaldehyde

14 g (0.16 mol) of manganese (IV) oxide are suspended in 100 ml of dichloromethane and then slowly combined with a solution of 5 g (0.04 mol) of 2-amino-benzylalcohol in 100 ml of dichloromethane. After 15 hours stirring the inorganic salts are suction filtered and the mother liquor is concentrated by evaporation.

Yield: 5 g (100% of theory),

R_{f} value: 0.51 (silica gel; dichloromethane)

b. 3-acetyl-1H-quinoline-2-one

5 g (0.04 mol) of 2-amino-benzaldehyde and 30 ml (0.23 mol) of ethyl acetoacetate are stirred for 1.5 hours at 160° C. using the water separator. Then the mixture is diluted with ether and the precipitated product is suction filtered.

Yield: 3.4 g (44% of theory),

R_{f} value: 0.72 (silica gel; cyclohexane/ethyl acetate=1:1)

c. 2-[4-(2-oxo-1,2-dihydro-quinolin-3-yl)-thiazol-2-ylamino-carbonyl]-benzoic acid

Prepared analogously to Example 1b from 3-acetyl-1H-quinolin-2-one and bromine in ethanol and subsequent reaction analogous to Example 1c with thiourea in ethanol. Then the 2-amino-4-(2-oxo-1,2-dihydro-quinolin-3-yl)-thiazole thus obtained is reacted with phthalic anhydride in pyridine analogously to Example 1d.

Yield: 68% of theory,

C₂₀H₁₃N₃O₄S (391.41)

Mass spectrum:

(M−H)⁻=390

### EXAMPLE 28

### 2-[4-(quinolin-3-yl)-thiazol-2-ylaminocarbonyl]-benzoic acid

Prepared analogously to Example 1d from 2-amino-4-(quinolin-3-yl)-thiazole and phthalic anhydride in pyridine.

Yield: 24% of theory,

C₂₀H₁₃N₃O₃S (375.41)

Mass spectrum:

(M−H)⁻=374

### EXAMPLE 29

### 3-[4-(naphthalin-2-yl)-thiazol-2-ylaminocarbonyl]-thiophene-2-carboxylic acid and 2-[4-(naphthalin-2-yl)-thiazol-2-ylaminocarbonyl]-thiophene-3-carboxylic acid

A mixture of 0.7 g (3.2 mmol) of 2-amino-4-(naphthalin-2-yl)-thiazole and 0.5 g (3.2 mmol) of thieno[2,3-c]furan-4,6-dione is stirred in 10 ml of 1,2-dichlorobenzene for 2 hours at 150° C. The precipitate is suction filtered and purified by chromatography, eluting with petroleum ether/ethyl acetate (6:4).

Yield: 0.2 g (15% of theory),

R_{f} value: 0.9 (silica gel; toluene/ethyl acetate/glacial acetic acid=50:45:5)

0.1 g (0.19 mmol) of the product thus obtained and 0.6 g (13.6 mmol) of lithium hydroxide are refluxed in 18 ml of tetrahydrofuran/water (4:5) for 2 hours. Then 10 ml of 20% potassium hydroxide solution and 10 ml of methanol are added and the mixture is refluxed for a further 3 hours. The solvent is distilled off and the residue purified by chromatography, eluting with dichloromethane/methanol (8:2).

Yield: 13 mg (18% of theory),

C₁₉H₁₂N₂O₃S₂ (380.45)

Mass spectrum:

(M−H)⁻=379

### EXAMPLE 30

### 4-bromo-2-[4-(naphthalin-2-yl)-thiazol-2-ylaminocarbonyl]-benzoic acid and 5-bromo-2-[4-(naphthalin-2-yl)-thiazol-2-ylaminocarbonyl]-benzoic acid

Prepared analogously to Example 1d from 2-amino-4-(naphthalin-2-yl)-thiazole and 4-bromo-phthalic anhydride in pyridine.

Yield: 0.26 g (16% of theory),

C₂₁H₁₃BrN₂O₃S (453.31)

Mass spectrum:

(M−H)⁻=451/53 (bromine isotope)

### EXAMPLE 31

### 3,4-Difluoro-2-[4-(naphthalin-2-yl)-thiazol-2-ylaminocarbonyl]-benzoic acid and 2,3-Difluoro-6-[4-(naphthalin-2-yl)-thiazol-2-ylaminocarbonyl]-benzoic acid

Prepared analogously to Example 1d from 2-amino-4-(naphthalin-2-yl)-thiazole and 5,6-difluoro-phthalic anhydride in pyridine.

Yield: 0.15 g (17% of theory),

C₂₁H₁₂F₂N₂O₃S (410.40)

Mass spectrum:

(M−H)⁻=409

### EXAMPLE 32

### 2-fluoro-6-[4-(naphthalin-2-yl)-thiazol-2-ylaminocarbonyl]-benzoic acid and 3-fluoro-2-[4-(naphthalin-2-yl)-thiazol-2-ylaminocarbonyl]-benzoic acid

Prepared analogously to Example 1d from 2-amino-4-(naphthalin-2-yl)-thiazole and 4-fluoro-phthalic anhydride in pyridine.

Yield: 0.66 g (51% of theory),

C₂₁H₁₃FN₂O₃S (392.41)

Mass spectrum:

(M−H)⁻=391

### EXAMPLE 33

### 4-nitro-2-[4-(naphthalin-2-yl)-thiazol-2-ylaminocarbonyl]-benzoic acid and 5-nitro-2-[4-(naphthalin-2-yl)-thiazol-2-ylaminocarbonyl]-benzoic acid

Prepared analogously to Example 1d from 2-amino-4-(naphthalin-2-yl)-thiazole and 4-nitro-phthalic anhydride in pyridine.

Yield: 0.38 g (27% of theory),

C_{21‘H}₁₃N₃O₅S (419.42)

Mass spectrum:

(M−H)⁻=418

### EXAMPLE 34

### 4-methyl-2-[4-(naphthalin-2-yl)-thiazol-2-ylaminocarbonyl]-benzoic acid and 5-methyl-2-[4-(naphthalin-2-yl)-thiazol-2-ylaminocarbonyl]-benzoic acid

Prepared analogously to Example 1d from 2-amino-4-(naphthalin-2-yl)-thiazole and 4-methyl-phthalic anhydride in pyridine.

Yield: 0.38 9 (30% of theory),

C₂₂H₁₆N₂O₃S (388.45)

Mass spectrum:

(M−H)⁻=387

### EXAMPLE 35

### 2-[4-(naphthalin-2-yl)-thiazol-2-ylaminocarbonyl]-cyclopent-1-ene-carboxylic acid

Prepared analogously to Example 1d from 2-amino-4-(naphthalin-2-yl)-thiazole and cyclopentene-1,2-dicarboxylic acid anhydride in pyridine.

Yield: 8% of theory,

C₂₀H₁₆N₂O₃S (364.43)

Mass spectrum:

M⁺=364

(M−H)⁻=363

### EXAMPLE 36

### 2-[4-(naphthalin-2-yl)-thiazol-2-ylaminocarbonyl]-nicotinic acid

Prepared analogously to Example 1d from 2-amino-4-(naphthalin-2-yl)-thiazole and pyridine-2,3-dicarboxylic acid anhydride in pyridine.

Yield: 19% of theory,

C₂₀H₁₃N₃O₃S (375.41)

Mass spectrum:

(M−H)⁻=374

### EXAMPLE 37

### 3-[4-(naphthalin-2-yl)-thiazol-2-ylaminocarbonyl]-isonicotinic acid and 4-[4-(naphthalin-2-yl)-thiazol-2-ylaminocarbonyl]-nicotinic acid

Prepared analogously to Example 1d from 2-amino-4-(naphthalin-2-yl)-thiazole and pyridine-3,4-dicarboxylic acid anhydride in pyridine.

Yield: 20% of theory,

C₂₀H₁₃N₃O₃S (375.41)

Mass spectrum: (M−H)⁻=374

### EXAMPLE 38

### 2-[4-(naphthalin-2-yl)-thiazol-2-ylaminocarbonyl]-cyclohex-1-ene-carboxylic acid

Prepared analogously to Example id from 2-amino-4-(naphthalin-2-yl)-thiazole and cyclohexene-1,2-dicarboxylic acid anhydride in pyridine.

Yield: 56% of theory,

C₂₁H₁₈N₂O₃S (378.45)

Mass spectrum:

(M−H)⁻=377

### EXAMPLE 39

### cis-2-[4-(naphthalin-2-yl)-thiazol-2-ylaminocarbonyl]-cyclo-propanecarboxylic acid

Prepared analogously to Example 1d from 2-amino-4-(naphthalin-2-yl)-thiazole and cyclopropane-1,2-dicarboxylic acid anhydride in pyridine.

Yield: 73% of theory,

C₁₈H₁₄N₂O₃S (338.38)

Mass spectrum:

M⁺=338

(M−H)⁻=337

### EXAMPLE 40

### (Z)-2,3-dimethyl-3-[4-(naphthalin-2-yl)-thiazol-2-yl-aminocarbonyl]-acrylic acid

Prepared analogously to Example 1d from 2-amino-4-(naphthalin-2-yl)-thiazole and 2,3-dimethyl-maleic anhydride in pyridine.

Yield: 8% of theory,

C₁₉H₁₆N₂O₃S (352.42)

Mass spectrum:

(M−H)⁻=351

### EXAMPLE 41

### 2-[4-(naphthalin-2-yl)-thiazol-2-yl-aminothiocarbonyl]-benzoic acid

1 g (2.57 mmol) of methyl 2-[4-(naphthalin-2-yl)-thiazol-2-ylaminocarbonyl]-benzoate and 0.52 g (1.28 mmol) of Lawesson's reagent are refluxed in 25 ml xylene for 15 hours. Then the mixture is concentrated by evaporation and the crude product is purified by chromatography, eluting with petroleum ether/ethyl acetate (8:2). The methyl 2-[4-(naphthalin-2-yl)-thiazol-2-ylaminothiocarbonyl]-benzoate thus obtained is then saponified analogously to Example 9b with lithium hydroxide in tetrahydrofuran/water.

Yield: 40 mg (6% of theory),

C₂₁H₁₄N₂O₂S₂ (390.49)

Mass spectrum:

(M−H)⁻=389

### EXAMPLE 42

### 4-[4-(naphthalin-2-yl]-thiazol-2-ylaminocarbonyl]-furan-3-carboxylic acid

a. 3,4-furandicarboxylic acid dichloride

1 g (6.4 mmol) of 3,4-furandicarboxylic acid are placed in 5 ml (68 mmol) of thionyl chloride and after the addition of 1 drop of dimethylformamide refluxed for 1 hour. Then the mixture is concentrated by evaporation and the residue is dissolved in 10 ml of tetrahydrofuran.

b. 4-[4-(naphthalin-2-yl)-thiazol-2-ylaminocarbonyl]-furan-3-carboxylic acid

1.4 g (6.4 mmol) of 2-amino-4-(naphthalin-2-yl)-thiazole are dissolved in 10 ml of tetrahydrofuran and combined with 2.7 ml (19.4 mmol) of triethylamine. Then the acid chloride prepared according to Example a) is added dropwise and the mixture is stirred for 25 hours. The solvent is distilled off and the residue is purified by chromatography, eluting with dichloromethane/ethanol (98:2). A solid product is thus obtained analogously to Example 29, which is reacted without any further purification.

Yield: 0.55 g (25% of theory),

R_{f} value: 0.75 (silica gel; dichloromethane/ethanol=19:1)

C₁₉H₁₀N₂O₃S (346.36)

Mass spectrum:

M⁺=346

0.55 g (1.6 mmol) of the solid product thus obtained and 10 ml (42.3 mmol) of 20% potassium hydroxide solution are refluxed in 16 ml of methanol for 6 hours. It is then concentrated by evaporation, the residue is diluted with water, the insoluble ingredients are suction filtered, the mother liquor is acidified with conc. hydrochloric acid and the precipitate is suction filtered.

Yield: 40 mg (7% of theory),

R_{f} value: 0.3 (silica gel; dichloromethane/ethanol=4:1)

C₁₉H₁₂N₂O₄S (364.38)

Mass spectrum:

M⁺=364

(M−H)⁻=363

### EXAMPLE 43

### N-[4-(naphthalin-2-yl)-thiazol-2-yl]-malonic acid monoamide,

a. monomethyl N-[4-(naphthalin-2-yl)-thiazol-2-yl]-malonate monoamide

0.87 g (3.8 mmol) of 2-amino-4-(naphthalin-2-yl)-thiazole and 1 ml (7.2 mmol) of triethylamine are placed in 10 ml of tetrahydrofuran and 0.4 ml (3.7 mmol) of monomethyl malonate chloride in 10 ml of tetrahydrofuran are added dropwise. After 25 hours' stirring the solvent is distilled off, the residue is distributed in ethyl acetate/water, the organic phase is separated off, dried over sodium sulphate and concentrated by evaporation.

Yield: 0.7 g (57% of theory),

R_{f} value: 0.6 (silica gel; dichloromethane/ethanol=9:1)

C₁₇H₁₄N₂O₃S (326.38)

Mass spectrum:

(M+H)⁺=327

(M+Na)⁺=349

(M−H)⁻=325

b. N-[4-(naphthalin-2-yl)-thiazol-2-yl]-malonic acid monoamide

Prepared analogously to Example 9b from monomethyl N-[4-(naphthalin-2-yl)-thiazol-2-yl]-malonate monoamide and lithium hydroxide in tetrahydrofuran/water.

Yield: 45% of theory,

C₁₆H₁₂N₂O₃S (312.35)

Mass spectrum:

(M−H)⁻=311

### EXAMPLE 44

### 3-[4-(naphthalin-2-yl)-thiazol-2-ylaminocarbonyl]-naphthaline-2-carboxylic acid

Prepared analogously to Example 1d from 2-amino-4-(naphthalin-2-yl)-thiazole and naphthaline[2,3-c]furan-1,3-dione in pyridine and subsequent reaction analogous to Example 42b with 20% potassium hydroxide solution in methanol.

Yield: 47% of theory),

C₂₅H₁₆N₂O₃S (424.48)

Mass spectrum:

(M−H)⁻=423

### EXAMPLE 45

### 5-[4-(naphthalin-2-yl)-thiazol-2-ylaminocarbonyl]-2H-[1,2,3]-triazole-4-carboxylic acid

Prepared analogously to 1d from 2-amino-4-(naphthalin-2-yl)-thiazole and methyl 5-chlorocarbonyl-2H-[1,2,3]triazole-4-carboxylate in tetrahydrofuran and subsequent saponification analogously to Example 9b with lithium hydroxide in tetrahydrofuran/water.

Yield: 22% of theory,

C₁₇H₁₁N₅O₃S (365.37)

Mass spectrum:

(M−H)⁻=364

### EXAMPLE 46

### 2-[4-(naphthalin-2-yl)-thiazol-2-ylaminocarbonyl]-cyclobut-1-enecarboxylic acid

Prepared analogously to Example 9b from methyl 2-[4-(naphthalin-2-yl)-thiazol-2-ylaminocarbonyl]-cyclobut-1-enecarboxylate and lithium hydroxide in tetrahydrofuran/water.

Yield: 100% of theory,

C₁₉H₁₄N₂O₃S (350.399)

Mass spectrum:

(M−H)⁻=349

### EXAMPLE 47

### 1-[4-(naphthalin-2-yl)-thiazol-2-ylaminocarbonyl]-cyclopropanecarboxylic acid

Prepared analogously to Example 9b from ethyl 1-[4-(naphthalin-2-yl)-thiazol-2-ylaminocarbonyl]-cyclopropanecarboxylate and lithium hydroxide in tetrahydrofuran/water.

Yield: 93% of theory,

C₁₈H₁₄N₂O₃S (338.388)

Mass spectrum:

(M−H)⁻=337

(M+H)⁺=339

### EXAMPLE 48

### trans-2-[4-(naphthalin-2-yl)-thiazol-2-ylaminocarbonyl]-cyclo-pentanecarboxylic acid

Prepared analogously to Example 9b from methyl 2-[4-(naphthalin-2-yl)-thiazol-2-ylaminocarbonyl]-cyclopentanecarboxylate and lithium hydroxide in tetrahydrofuran/water.

Yield: 67% of theory,

C₂₀H₁₈N₂O₃S (366.442)

Mass spectrum:

(M−H)⁻=365

(M+H)⁺=367

(M+Na)⁺=389

### EXAMPLE 49

### 2-[4-(naphthalin-2-yl)-thiazol-2-ylamino]-nicotinic acid

a. methyl 2-[4-(naphthalin-2-yl)-thiazol-2-ylamino]-nicotinate

A mixture of 0.5 g (1.5 mmol) of methyl 2-chloro-nicotinate, 0.3 g (1.9 mmol) of 2-amino-4-(naphthalin-2-yl)-thiazole, 1.3 g (3.99 mmol) of caesium carbonate, 15 mg (0.067 mmol) of palladium(II)acetate and 40 mg (0.064 mmol) of 2,2′-bis-(diphenylphosphino)-1,1′-binaphthyl is stirred in 10 ml of xylene for 6 hours at 120° C. Then the solvent is distilled off, the residue is distributed in dichloromethane/water, the organic phase is separated off and concentrated by evaporation. The residue is purified by chromatography, eluting with dichloromethane/ethanol (99:1).

Yield: 0.2 g (41% of theory),

R_{f} value: 0.6 (silica gel; dichloromethane/ethanol=19:1)

C₂₀H₁₅N₃O₂S (361.43)

Mass spectrum:

(M+H)⁺=362

(M+Na)⁺=384

b. 2-[4-(naphthalin-2-yl)-thiazol-2-ylamino]-nicotinic acid

Prepared analogously to Example 9b from methyl 2-[4-(naphthalin-2-yl)-thiazol-2-ylamino]-nicotinate and lithium hydroxide in tetrahydrofuran/water.

Yield: 91% of theory,

C₁₉H₁₃N₃O₂S (347.398)

Mass spectrum:

M⁺=347

(M−H)⁻=346

### EXAMPLE 50

### 2-[4-(naphthalin-2-yl)-pyrimidin-2-ylamino]-benzoic acid

a. 2-chloro-4-(naphthalin-2-yl)-pyrimidine

Prepared analogously to Example 18a from 2,4-dichloro-pyrimidine, 2-naphthylboric acid, tetrakis-(triphenylphosphine)-palladium, 2,2′-bis-(diphenylphosphino)-1,1′-binaphthyl and sodium carbonate in dimethoxyethane.

Yield: 37% of theory,

R_{f} value: 0.6 (silica gel; petroleum ether/ethyl acetate=6:4)

C₁₄H₉ClN₂ (240.69)

Mass spectrum:

M⁺=240/242 (chlorine isotope)

(M+H)⁺=241/243

b. 2-[4-(naphthalin-2-yl)-pyrimidin-2-ylamino]-benzoic acid

A mixture of 0.2 9 (0.83 mmol) of 2-chloro-4-(naphthalin-2-yl)-pyrimidine and 0.1 g (0.83 mmol) of anthranilic acid is refluxed in 10 ml of ethanol and 0.1 ml of conc. hydrochloric acid for 9 hours. Then it is diluted with water and the precipitate is suction filtered. The product thus obtained is reacted analogously to Example 42b with 20% potassium hydroxide solution in methanol.

Yield: 42% of theory,

C₂₁H₁₅N₃O₂ (341.373)

Mass spectrum:

(M−H)⁻=340

### EXAMPLE 51

### 2-[6-(naphthalin-2-yl)-pyridin-2-ylamino]-benzoic acid

Prepared analogously to Example 9b from methyl 2-[6-(naphthalin-2-yl)-pyridin-2-ylamino]-benzoate and lithium hydroxide in tetrahydrofuran/water.

Yield: 4% of theory,

C₂₂H₁₆N₂O₂ (340.385)

Mass spectrum:

M⁺=340

### EXAMPLE 52

### 2-[3-(naphthalin-2-yl)-phenylamino]-nicotinic acid

a. 2-(3-nitro-phenyl)-naphthaline

Prepared analogously to Example 18a from 1-bromo-3-nitrobenzene, 2-naphthylboric acid, tetrakis-(triphenylphosphine)-palladium, 2,2′-bis-(diphenylphosphino)-1,1′-binaphthyl and sodium carbonate in dimethyoxyethane.

Yield: 81% of theory,

R_{f} value: 0.5 (silica gel; petroleum ether/ethyl acetate=9:1)

b. 3-(naphthalin-2-yl)-aniline

To a solution of 1.6 g (6.4 mmol) of 2-(3-nitro-phenyl)-naphthaline in 25 ml of pyridine are added 4.5 g (25.8 mmol) of sodium dithionite in 15 ml of water and the mixture is stirred for 1 hour at 55° C. Then 50 ml of semisaturated sodium carbonate solution are added and the precipitate is suction filtered. The mother liquor is extracted with dichloromethane, the combined organic extracts are dried and concentrated by evaporation.

Yield: 0.8 g (57% of theory),

R_{f} value: 0.6 (silica gel; petroleum ether/ethyl acetate=5:5)

c. 2-[3-(naphthalin-2-yl)-phenylamino]-nicotinic acid

Prepared analogously to Example 49a from 3-(naphthalin-2-yl)-aniline, methyl 2-chloro-nicotinate, caesium carbonate, 2,2′-bis-(diphenylphosphino)-1,1′-binaphthyl and palladium (II) acetate in xylene and subsequent saponification of the methyl 2-[3-(naphthalin-2-yl)-phenylamino]-nicotinate thus obtained analogously to Example 9b with lithium hydroxide in tetrahydrofuran/water.

Yield: 47% of theory,

C₂₂H₁₆N₂O₂ (340.385)

Mass spectrum:

M⁺=340

(M−H)⁻=339

(M+H)⁺=341

### EXAMPLE 53

### 2-[3-(naphthalin-2-yl)-phenylamino]-benzoic acid

Prepared analogously to Example 49a from 3-(naphthalin-2-yl)-aniline, methyl 2-iodo-benzoate, caesium carbonate, 2,2′-bis-(diphenylphosphino)-1,1′-binaphthyl and palladium (II) acetate in xylene and subsequent saponification of the methyl 2-[3-(naphthalin-2-yl)-phenylamino]-benzoate thus obtained analogously to Example 9b with lithium hydroxide in tetrahydrofuran/water.

Yield: 43% of theory,

C₂₃H₁₇NO₂ (339.397)

Mass spectrum:

(M−H)³¹ =338

### EXAMPLE 54

### 2-[4-methyl-6-(naphthalin-2-yl)-pyrimidin-2-ylamino]-5-nitro-benzoic acid

a. 2-amino-4-methyl-6-(naphthalin-2-yl)-pyrimidine

Prepared analogously to Example 18a from 2-amino-4-chloro-6-methylpyrrolidone, 2-naphthalineboric acid, tetrakis-(triphenylphosphine)-palladium, 2,2′-bis-(diphenylphosphino)-1,1′-binaphthyl and sodium carbonate in dimethyoxyethane.

Yield: 55% of theory,

R_{f} value: 0.4 (silica gel; petroleum ether/ethyl acetate=4:6)

C₁₅H₁₃N₃ (235.29)

Mass spectrum:

(M+H)⁺=236

b. 2-[4-methyl-6-(naphthalin-2-yl)-pyrimidin-2-ylamino]-5-nitro-benzoic acid

Prepared analogously to Example 49a from 2-amino-4-methyl-6-(naphthalin-2-yl)-pyrimidine, methyl 2-bromo-5-nitro-benzoate, caesium carbonate, 2,2′-bis-(diphenylphosphino)-1,1′-binaphthyl and palladium (II) acetate in xylene and subsequent saponification of the methyl 2-[4-methyl-6-(naphthalin-2-yl)-pyrimidin-2-ylamino]-5-nitro-benzoate thus obtained analogously to Example 9b with lithium hydroxide in tetrahydrofuran/water.

Yield: 48% of theory,

C₂₂H₁₆N₄O₄ (400.397)

Mass spectrum:

(M−H)⁻=399

### EXAMPLE 55

### 5-amino-2-[4-methyl-6-(naphthalin-2-yl)-pyrimidin-2-ylamino]-benzoic acid

a. methyl 2-[4-methyl-6-(naphthalin-2-yl)-pyrimidin-2-ylamino]-5-amino-benzoate

0.4 g (0.91 mmol) of methyl 2-[4-methyl-6-(naphthalin-2-yl)-pyrimidin-2-ylamino]-5-nitro-benzoate are dissolved in 50 ml of tetrahydrofuran and after the addition of 250 mg of 10% palladium on charcoal hydrogenated for 2 hours with hydrogen. The catalyst is filtered off and the mother liquor is concentrated by evaporation. The crude product is purified by chromatography, eluting with petroleum ether/ethyl acetate (6:4).

Yield: 0.3 g (71% of theory),

R_{f} value: 0.35 (silica gel; petroleum ether/ethyl acetate=1:1)

C₂₃H₂₀N₄O₂ (384.44)

Mass spectrum:

(M+H)⁺=385

b. 5-amino-2-[4-methyl-6-(naphthalin-2-yl)-pyrimidin-2-yl-amino]-benzoic acid

Prepared analogously to Example 9b from methyl 5-amino-2-[4-methyl-6-(naphthalin-2-yl)-pyrimidin-2-ylamino]-benzoate and lithium hydroxide in tetrahydrofuran/water.

Yield: 39% of theory,

C₂₂H₁₈N₄O₂ (370.41)

Mass spectrum:

(M−H)⁻=369

### EXAMPLE 56

### 5-acetylamino-2-[4-methyl-6-(naphthalin-2-yl)-pyrimidin-2-yl-amino]-benzoic acid

0.2 g (0.54 mmol) of methyl 2-[4-methyl-6-(naphthalin-2-yl)-pyrimidin-2-ylamino]-5-amino-benzoate are dissolved in 5 ml of pyridine and after the addition of 0.1 g (0.63 mmol) of acetyl chloride stirred for 4 hours. Then the solvent is distilled off, the residue is taken up in 12 ml of water and 16 ml of tetrahydrofuran and after the addition of 8 ml (8 mmol) of 1 molar lithium hydroxide solution the mixture is stirred for a further 6 hours. Then it is neutralised with hydrochloric acid and concentrated by evaporation. The crude product is purified by chromatography, eluting with ethyl acetate/ethanol (7:3). Yield: 0.1 g (58% of theory),

C₂₄H₂₀N₄O₃ (412.45)

Mass spectrum:

(M−H)⁻=411

(M+H)⁺=413

### EXAMPLE 57

### 2-[4-(naphthalin-2-yl)-thiazol-2-ylamino]-5-nitro-benzoic acid

Prepared analogously to Example 9b from methyl 2-[4-(naphthalin-2-yl)-thiazol-2-ylamino]-5-nitro-benzoate and lithium hydroxide in tetrahydrofuran/water.

Yield: 39% of theory,

C₂₀H₁₃N₃O₄S (391.41)

Mass spectrum:

(M−H)⁻=390

### EXAMPLE 58

### 5-acetylamino-2-[4-(naphthalin-2-yl)-thiazol-2-ylamino]-benzoic acid

Prepared analogously to Example 56 from methyl 5-amino-2-[4-(naphthalin-2-yl)-thiazol-2-ylamino]-benzoate and acetyl chloride in pyridine and subsequent saponification with lithium hydroxide in tetrahydrofuran/water.

Yield: 49% of theory,

C₂₂H₁₇N₃O₃S (403.46)

Mass spectrum:

(M−H)⁻=402

### EXAMPLE 59

### 2-[4-(naphthalin-2-yl)-pyrimidin-2-ylamino]-4-(pyrrolidin-1-ylaminocarbonyl)-benzoic acid

Prepared analogously to Example 9b from methyl 2-[4-(naphthalin-2-yl)-pyrimidin-2-ylamino]-4-(pyrrolidin-1-ylaminocarbonyl)-benzoate and sodium hydroxide in methanol.

Yield: 32% of theory,

C₂₆H₂₃N₅O₃ (453.50)

Mass spectrum:

(M−H)⁻=452

(M+H)⁺=454

### EXAMPLE 60

### 2-[4-(naphthalin-2-yl)-thiazol-2-ylcarbonylamino]-terephthalic acid

Prepared analogously to Example 9b from dimethyl 2-[4-(naphthalin-2-yl)-thiazol-2-ylcarbonylamino]-terephthalate and potassium hydroxide in methanol.

Yield: 32% of theory,

C₂₂H₁₄N₂O₅S (418.43)

Mass spectrum:

(M−H)⁻=417

### EXAMPLE 61

### 2-[4-(1,3-dihydro-isoindol-2-yl)-pyrimidin-2-ylamino]-benzoic acid

a. 2-chloro-4-(2,3-dihydro-1H-isoindol-2-yl)-pyrimidine

A mixture of 0.5 g (3.35 mmol) of 2,4-dichloro-pyrimidine, 0.5 g (3.2 mmol) of 2,3-dihydro-1H-isoindol-hydrochloride and 0.6 ml (3.4 mmol) of N-ethyl-diisopropylamine is stirred in 40 ml dichloromethane for 3 hours. Then the mixture is concentrated by evaporation, the residue is distributed in ethyl acetate/water, the organic phase is separated off and concentrated by evaporation.

Yield: 0.4 g (55% of theory),

R_{f} value: 0.4 (silica gel; dichloromethane/ethanol=19:1)

b. 2-[4-(2,3-dihydro-1H-isoindol-2-yl)-pyrimidin-2-ylamino]-benzoic acid

Prepared analogously to Example 49a from 2-chloro-4-(2,3-dihydro-1H-isoindol-2-yl)-pyrimidine, methyl anthranilate, caesium carbonate, palladium(II)acetate and 2,2′-bis-(diphenylphosphino)-1,1′-binaphthyl in xylene and subsequent saponification of the methyl 2-[4-(2,3-dihydro-1H-isoindol-2-yl)-pyrimidin-2-ylamino]-benzoate thus obtained with lithium hydroxide in tetrahydrofuran/water analogously to Example 9b.

Yield: 4% of theory,

C₁₉H₁₆N₄O₂ (332.36)

Mass spectrum:

(M−H)⁻=331

(M+H)⁺=333

### EXAMPLE 62

### 2-[3-(naphthalin-2-yl)-phenylcarbonylamino]-benzoic acid

Prepared analogously to Example 9b from methyl 2-[3-(naphthalin-2-yl)-phenylcarbonylamino]-benzoate and sodium hydroxide in tetrahydrofuran/water.

Yield: 50% of theory,

C₂₄H₁₇NO₃ (367.41)

Mass spectrum:

(M−H)⁻=366

(M+Na)⁺=390

M⁺=367

The following compounds may be prepared analogously to Example 62:

(1) 2-[4-(naphthalin-2-yl)-pyrimidin-2-ylamino]-4-(methyl-aminocarbonyl)-benzoic acid

(2) 2-[4-(naphthalin-2-yl)-pyrimidin-2-ylamino]-4-(ethylamino-carbonyl)-benzoic acid

(3) 2-[4-(naphthalin-2-yl)-pyrimidin-2-ylamino]-4-(propyl-aminocarbonyl)-benzoic acid

(4) 2-[4-(3-bromo-4-chloro-phenyl)-thiazol-2-ylaminocarbonyl]-benzoic acid

(5) 2-[4-(4-bromo-3-chloro-phenyl)-thiazol-2-ylaminocarbonyl]-benzoic acid

(6) 2-[4-(3,4-Dibromo-phenyl)-thiazol-2-ylaminocarbonyl]-benzoic acid

### EXAMPLE 63

Tablets Containing 50 mg of Active Substance

| | | |
|---|---|---|
| Active substance | 50.0 | mg |
| Calcium phosphate | 70.0 | mg |
| Lactose | 40.0 | mg |
| Corn starch | 35.0 | mg |
| Polyvinylpyrrolidone | 3.5 | mg |
| Magnesium stearate | 1.5 | mg |
| | 200.0 | mg |

Preparation:

The active substance, CaHPO₄, lactose and corn starch are evenly moistened with an aqueous PVP solution. The mass is passed through a 2-mm screen, dried in a circulating air drier at 50° C. and screened again.

After the lubricant has been mixed in, the granules are compressed in a tablet-making machine.

### EXAMPLE 64

Coated Tablets Containing 50 mg of Active Substance

| | | |
|---|---|---|
| Active substance | 50.0 | mg |
| Lysine | 25.0 | mg |
| Lactose | 60.0 | mg |
| Corn starch | 34.0 | mg |
| Gelatine | 10.0 | mg |
| Magnesium stearate | 1.0 | mg |
| | 180.0 | mg |

Preparation:

The active substance is mixed with the excipients and moistened with an aqueous gelatine solution. After screening and drying, the granules are mixed with magnesium stearate and compressed to form tablet cores.

The cores thus produced are covered with a coating by known methods. The coating suspension or solution may have colouring added to it.

### EXAMPLE 65

Coated Tablets Containing 100 mg of Active Substance

| | | |
|---|---|---|
| Active substance | 100.0 | mg |
| Lysine | 50.0 | mg |
| Lactose | 86.0 | mg |
| Corn starch | 50.0 | mg |
| Polyvinylpyrrolidone | 2.8 | mg |
| Microcrystalline cellulose | 60.0 | mg |
| Magnesium stearate | 1.2 | mg |
| | 350.0 | mg |

Preparation:

The active substance is mixed with the excipients and moistened with an aqueous PVP solution. The moist mass is passed through a 1.5 mm screen and dried at 45° C. After drying, the mass is screened again and the magnesium stearate is added. This mixture is compressed to form tablet cores. The cores thus produced are covered with a coating by known methods. The coating suspension or solution may have colouring added to it.

### EXAMPLE 66

Capsules Containing 250 mg of Active Substance

| | | |
|---|---|---|
| Active substance | 250.0 | mg |
| Corn starch | 68.5 | mg |
| Magnesium stearate | 1.5 | mg |
| | 320.0 | mg |

Preparation:

Active substance and corn starch are mixed together and moistened with water. The moist mass is screened and dried. The dry granules are screened and mixed with magnesium stearate. The final mixture is packed into size 1 hard gelatine capsules.

1. Carboxylic acid derivatives of general formula
R₁—A—B—R₂ (1)
wherein
R₁ denotes a phenyl, phenyl-C₁₋₃-alkyl, phenyl-C₂₋₄-alkenyl or naphthyl group, wherein in each case the aromatic moieties may be mono- or disubstituted by a fluorine, chlorine, bromine or iodine atom, by a C₁₋₃-alkyl or C₁₋₃-alkoxy group, while the substituents may be identical or different,
a phenyl group, to which an n-propylene, n-butylene, methylenedioxy or ethylenedioxy bridge is fused via two adjacent carbon atoms,
a phenyl group, to which a 5-membered heteroaromatic group is fused via two adjacent carbon atoms, which contains, in the heteroaromatic moiety,
an imino group optionally substituted by a C₁₋₃-alkyl group, an oxygen or sulphur atom,
an imino group optionally substituted by a C₁₋₃-alkyl group and an oxygen, sulphur or nitrogen atom,
an imino group optionally substituted by a C₁₋₃-alkyl group and two nitrogen atoms or
an oxygen or sulphur atom and two nitrogen atoms,
a pyridinyl or pyronyl group optionally substituted by a C₁₋₃-alkyl group, to which a phenyl ring may be fused in each case via two adjacent carbon atoms, while in the abovementioned pyridine ring additionally a methine group in the 2 or 4 position may be replaced by a hydroxymethine group,
A denotes a phenylene group optionally substituted by a C₁₋₃-alkyl group, wherein in the aromatic moiety one, two or three methine groups may be replaced by nitrogen atoms, or
a 5-membered heteroarylene group optionally substituted by a C₁₋₃-alkyl group, while the heteroaromatic moiety is as hereinbefore defined,
B denotes an —HN, —NH—CO, —CO—NH, —NH—CS or —CS—NH group, wherein the —NH group may be substituted in each case by a C₁₋₃-alkyl group, and
R₂ denotes a C₃₋₇-cycloalkyl or C₄₋₇-cycloalkenyl group substituted by a carboxy group,
a phenyl or naphthyl group substituted by a carboxy group, wherein in each case the aromatic moiety may be replaced by a nitro, amino, C₁₋₃-alkylamino, di-(C₁₋₃-alkyl)-amino, C₁₋₃-alkanoylamino, N-(C₁₋₃-alkyl)-C₁₋₃-alkanoylamino or carboxy group, by an aminocarbonyl or C₁₋₃-alkylaminocarbonyl group, wherein in each case the hydrogen atom of the aminocarbonyl group is monosubstituted by a C₁₋₃-alkyl or C₃₋₇-cycloalkyleneimino group, or is mono- or disubstituted by a fluorine, chlorine, bromine or iodine atom, by a C₁₋₃-alkyl or C₁₋₃-alkoxy group, while the substituents may be identical or different,
a 5- or 6-membered heteroaryl group substituted by a carboxy group, while the 5-membered heteroaryl group is as hereinbefore defined and
the 6-membered heteroaryl group contains one or two nitrogen atoms,
or a straight-chain or branched C₁₋₆-alkyl or C₂₋₆-alkenyl group substituted by a carboxy group,
while the carboxy groups mentioned in the definition of the abovementioned groups may additionally be replaced by a group which is converted in vivo into a carboxy group or is negatively charged under physiological conditions,
and the imino or amino group mentioned in the definition of the abovementioned groups may be substituted by a group which can be cleaved in vivo,
the isomers and the salts thereof.

2. Carboxylic acid derivatives of general formula I according to claim 1, wherein
R₁ denotes a phenyl group which may be substituted by a chlorine, bromine or iodine atom or may be mono- or disubstituted by a methyl or methoxy group, while the substituents may be identical or different,
a phenylvinyl, benzothiophenyl or naphthyl group,
a phenyl group to which an n-propylene, n-butylene, methylenedioxy or ethylenedioxy bridge is fused via two adjacent carbon atoms,
an pyridinyl or pyronyl group optionally substituted by a methyl group, to which a phenyl ring is fused in each case via two adjacent carbon atoms, while in the abovementioned pyridine ring a methine group in the 2 or 4 position may additionally be replaced by a hydroxymethine group,
A denotes a phenylene, furanylene, thiophenylene, thiazolylene, imidazolylene, thiadiazolylene, pyridinylene or pyrimidylene group optionally substituted by a methyl group with the proviso that linking to the adjacent groups R₁ and B does not take place via the o position of the abovementioned aromatic groups,
B denotes an —HN, —NH—CO, —CO—NH, —NH—CS or —CS—NH group, wherein the —NH group may be substituted in each case by a methyl group, and
R₂ denotes a C₃₋₆-cycloalkyl or C₄₋₆-cycloalkenyl group substituted by a carboxy group,
a phenyl group substituted by a carboxy group which is monosubstituted in the phenyl moiety by a nitro, amino, acetylamino, carboxy, aminocarbonyl or pyrrolidinoaminocarbonyl group or is mono- or disubstituted by a fluorine, chlorine, bromine or iodine atom or by a methyl or methoxy group, while the substituents may be identical or different,
a carboxy-substituted naphthyl, furanyl, thiophenyl, triazolyl or pyridinyl group,
an aminocarbonylmethyl group or a carboxy-substituted methyl or 1,2-dimethylvinyl group,
the isomers and the salts thereof.

3. Carboxylic acid derivatives of general formula I according to claim 1, wherein
R₁, R₂ and A are defined as in claim 2, and
B denotes an —NH or —NH—CO group, while the —NH—CO group is linked to the group R₂ via the —CO group,
the isomers and the salts thereof.

4. Carboxylic acid derivatives of general formula I according to claim 1, wherein
R₁ denotes a phenyl group optionally mono- or disubstituted by a chlorine, bromine or iodine atom, while the substituents may be identical or different,
a naphthyl or (2-oxo-2H-chromen-3-yl) group,
A denotes a 1,3-phenylene, 2,5-thiazolylene, 2,4-pyridinylene, 2,6-pyridinylene or 2,4-pyrimidylene group,
B denotes an —NH or —NH—CO group, while the —NH—CO group is linked to the group R₂ via the —CO group,
R₂ denotes a 2-carboxy-cyclopent-2-enyl, 2-carboxy-cyclohex-2-enyl, 3-carboxy-thien-2-yl or 2-carboxy-1,2-dimethyl-vinyl group or
a 2-carboxy-phenyl group optionally monosubstituted by a fluorine, chlorine or bromine atom or by a methyl or nitro group,
the isomers and the salts thereof.

5. The following carboxylic acid derivatives of general formula I according to claim 1:
(a) 2-[4-(naphthalin-2-yl)-thiazol-2-ylaminocarbonyl]-benzoic acid,
(b) 2-[4-(naphthalin-2-yl)-thiazol-2-ylaminocarbonyl]-cyclopent-1-ene-carboxylic acid and
(c) 2-[4-(naphthalin-2-yl)-pyrimidin-2-ylamino]-benzoic acid
as well as their salts.

6. Physiologically acceptable salts of the compounds according to claim 1.

7. Pharmaceutical compositions containing a compound according to claim 1 optionally together with one or more inert carriers and/or diluents.

8. Pharmaceutical compositions containing a compound according to claim 6 optionally together with one or more inert carriers, and/or diluents.

9. Use of a compound according to claim 1 for preparing a pharmaceutical composition having an inhibiting effect on telomerase.

10. Use of a compound according to a salt according to claim 6 for preparing a pharmaceutical composition having an inhibiting effect on telomerase.
